(19) United States
(12) Patent Application Publication
(10) Pub. No.: US 2021/0251947 A1
(43) Pub. Date: Aug. 19, 2021

(54) STABLE FORMULATIONS OF DRONABINOL

(71) Applicant: TRYAGx Labs Inc., Midlothian, VA (US)

(72) Inventor: Nazar S. Elkarim, Midlothian, VA (US)

(21) Appl. No.: 17/171,299

(22) Filed: Feb. 9, 2021

Related U.S. Application Data
(60) Provisional application No. 62/972,174, filed on Feb. 10, 2020.

(51) Int. Cl. A61K 31/352; A61K 47/10; A61K 47/44; A61K 47/14; A61K 47/24; A61K 47/22; A61K 9/48; A61K 9/00; A61J 1/03

(52) U.S. Cl. CPC A61K 31/352; A61K 47/10; A61K 47/44; A61K 47/14; A61K 47/24; A61K 47/22; A61J 1/03; A61K 9/4833; A61K 9/0053; A61K 9/4875; A61K 9/4866; A61K 9/4858; A61J 1/035; A61K 9/4825

(57) ABSTRACT

Described herein are formulations, methods of manufacturing, and methods of treatment using formulations of cannabinoids that are stable at room temperature for at least about one to two years. In one embodiment, the composition is an oxidatively stable formulation of dronabinol.

### CROSS REFERENCE TO RELATED APPLICATIONS

This application claims priority to U.S. Provisional Patent Application No. 62/972,174, filed on Feb. 10, 2020, which is incorporated by reference herein in its entirety.

### TECHNICAL FIELD

Described herein are formulations, methods of manufacturing, and methods of treatment using formulations of cannabinoids that are stable at room temperature for at least about one to two years. In one embodiment, the composition is an oxidatively stable formulation of dronabinol.

### BACKGROUND

Δ-9-tetrahydrocannabinol (also known as Δ⁹-THC, THC, or dronabinol) is a naturally occurring compound and is the primary active ingredient in the controlled substance marijuana. Marijuana refers to the dried flowers and leaves of the plant Cannabis sativa. Cannabis contain several compounds called cannabinoids (including dronabinol), that may help patients with certain disease conditions. Dronabinol has been approved by the Food and Drug Administration (FDA) for the control of nausea and vomiting associated with chemotherapy and for appetite stimulation of AIDS patients suffering from wasting syndrome. Synthetic dronabinol has been utilized as a pharmaceutically active ingredient, and Cannabis-based medicines using botanical sources of Cannabis rather than synthetic THC are known in the art.

Dronabinol (Δ⁹-THC) is a cannabinoid designated chemically as (6aR,10aR)-6,6,9-trimethyl-3-pentyl-6a,7,8,10a-tetrahydro-6H-benzo[c]chromen-1-ol. Dronabinol has the following structural formula (1):

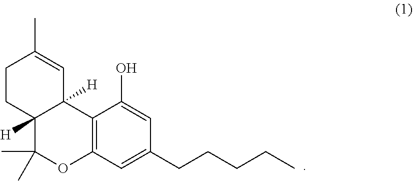 [SMILES: CCCCCc1cc(O)c2c(c1)OC(C)(C)[C@@H]1CCC(C)=C[C@@H]21]

Dronabinol has the chemical formula C₂₁H₃₀O₂ and a molecular weight of 314.47. Dronabinol is a light-yellow resinous oil that is sticky at room temperature and hardens upon refrigeration. Dronabinol is insoluble in water and is typically formulated in sesame oil or ethanol. It has a pKₐ of 10.6 and an octanol-water partition coefficient: 6,000:1 at pH 7.

Dronabinol has been commercially available in the U.S. since 1985 under the tradename MARINOL® (AbbVie Inc.). MARINOL® is a soft gelatin capsule containing a sesame oil solution of synthetic Δ⁹-THC, dronabinol. Upon oral administration, the gelatin dissolves, releasing the drug. The dronabinol dissolved in sesame oil is then absorbed during its passage through the gastrointestinal tract. The MARINOL® soft gelatin dosage form is unstable at room temperature and the product must be stored under refrigerated (2-8° C.) or cool (8-15° C.) conditions. See MARINOL® package label 2017, AbbVie Inc., which is incorporated by reference for such teachings.

A liquid formulation of dronabinol for oral administration was approved in 2017 under the tradename Syndros®. See Syndros® package label 2016, Insys Therapeutics, which is incorporated by reference for such teachings. Syndros® must also be stored under refrigerated conditions prior to use. Upon opening, the bottle can be stored at room temperature (e.g., between 20-25° C.) for up to 28 days and any unused solution should be discarded thereafter.

Dronabinol's instability is believed to result from oxidation, which is increased at room temperature. Dronabinol is oxidized to cannabinol and cannabidiol in the presence of oxygen. Mechoulam R., Psychotropic Agents, Handbook Exp. Pharmacol. 55: 119-134 (1982). Dronabinol has been reported to rapidly degrade when exposed to heat, high humidity, or high moisture conditions. Such conditions are typical for pharmaceutical formulations. In addition, glycerol is a major component in the gelatin shell that comprises the soft gelatin capsule. It has been reported that because lipophilic compounds such as dronabinol are soluble in glycerol, these compounds migrate into the shell of soft gelatin capsules. Armstrong et al., J. Pharm. Pharmacol. 36(6):361-365 (1984). The large amount of glycerol present in soft gelatin may also lead to increased permeability of the gelatin shell to oxygen. Cade et al., Acta Pharm. Technol. 33:97-100 (1987); Hom et al., J. Pharm. Sci. 64(5):851-7 (1975). In addition, the large amount of glycerol in soft gelatin capsules may increase sensitivity of formulations to heat and humidity. Bauer, in: Die Kapsel. Stuttgart: Wissenschaftliche Verlags GmbH. Editors: Fahrig W, Hofer U H, 58-82 (1983).

The need to store dronabinol formulations under refrigerated conditions is a major disadvantage for these pharmaceutical products. Accordingly, there is a need for stable dronabinol dosage forms that do not require refrigerated storage conditions.

### SUMMARY

One embodiment described herein is a pharmaceutical composition comprising: one or more cannabinoids; one or more solvents or vehicles; and one or more low melting hard lipids; wherein the composition has enhanced oxidative stability and is stable at ambient temperature for at least 1-2 years. In one aspect, the composition further comprises one or more antioxidants. In another aspect, the composition further comprises one or more pharmaceutically acceptable excipients. In another aspect, the cannabinoid comprises one or more of: Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); Δ⁹-tetrahydrocannabivarin (Δ⁹-THCBV); tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-NE); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); tetrahydrocannabibutoic acid (Δ⁹-THCBA); Δ⁸-tetrahydrocannabibutolic acid (Δ⁸-THCBA); Δ⁹-tetrahydrocannabibutol naphtoylester (Δ⁹-THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof. In another aspect, the cannabinoid comprises a tetrahydrocannabinol. In another aspect, the cannabinoid is dronabinol, Δ⁹-tetrahydrocannabinol (Δ⁹-THC). In another aspect, the solvent or vehicle comprises one or more oils comprising sesame, sunflower, soybean, or vegetable oils. In another aspect, low melting hard lipid comprises one or more polyethylene glycol glycerides. In another aspect, the low melting hard lipid comprises a polyethylene glycol glyceride with an HLB value of about 1 and a melting point of about 42° C. to about 46° C. (Gelucire® 43/01) or an HLB value of about 13 and a melting point of about 50° to about 56° C. (Gelucire® 43/01). In another aspect, the cannabinoid is suspended in sesame oil, propylene glycol dicaprylate/dicaprate (Miglyol® 840), or ethanol. In another aspect, the cannabinoid is suspended in sesame oil. In another aspect, the antioxidant comprises one or more of butylated hydroxyanisole (BHA), butylated hydroxytoluene (BHT), tocopherol, D/L-α-tocopherol, Vitamin E, vitamin E tocopherol, Vitamin E-TPGS, propylene gallate lecithin, sesamin, sesamol, sesamolin, ascorbic acid, ascorbyl palmitate, sodium ascorbate, fumaric acid, malic acid, sodium metabisulphite, or disodium ethylenediaminetetracetic acid (EDTA), or combinations thereof. In another aspect, the antioxidant comprises one or more of butylated hydroxytoluene (BHT) BHT, tocopherol, D/L-α-tocopherol, Vitamin E, Vitamin E-TPGS, propylene gallate, lecithin, or combinations thereof. In another aspect, the composition comprises: 1-10% by mass of one or more cannabinoids; 60-85% by mass of one or more solvents or vehicles; 7.5-20% by mass of one or more low-melting hard lipids; and 0.1-20% by mass of one or more antioxidants. In another aspect, the composition comprises: 2.5 mg, 5 mg, or 10 mg of the cannabinoid. In another aspect, the composition is encapsulated in a soft gelatin capsule. In another aspect, the soft gelatin capsule comprises: 25-50% by mass of one or more film-forming polymers; 15-25% by mass of one or more plasticizers; 20-40% by mass of a solvent; and optionally, one or more opacifying agents, coloring agents, or pharmaceutically acceptable excipients. In another aspect, the soft gelatin capsule comprises: 25-50% of gelatin; 15-25% of sorbitol; 20-40% of water; and optionally, one or more opacifying agents, coloring agents, or pharmaceutically acceptable excipients.

Another embodiment described herein is a stable pharmaceutical dosage form comprising a soft gelatin capsule encapsulating: 1-10% by mass of one or more cannabinoids; 60-85% by mass of one or more of sesame, sunflower, soybean, or vegetable oils; 7.5-20% by mass of one or more low-melting hard lipid comprising a polyethylene glycol glyceride with an HLB value of about 1 and a melting point of about 42° C. to about 46° C. (Gelucire® 43/01) or an HLB value of about 13 and a melting point of about 50° to about 56° C. (Gelucire® 43/01)s; and 0.1-20% by mass of one or more antioxidants comprising comprises one or more of butylated hydroxytoluene (BHT) BHT, tocopherol, D/L-α-tocopherol, Vitamin E, Vitamin E-TPGS, propylene gallate, lecithin, or combinations thereof; wherein the composition has enhanced oxidative stability and is stable at ambient temperature for at least 1-2 years. In one aspect, the composition comprises 2.5 mg, 5 mg, or 10 mg of dronabinol. In another aspect, the soft gelatin capsule comprises: 25-50% of gelatin; 15-25% of sorbitol; 20-40% of water; and optionally, one or more opacifying agents, coloring agents, or pharmaceutically acceptable excipients. In another aspect, the composition is bioequivalent to dronabinol, capsules for oral use (MARINOL®). In another aspect, the dosage form releases 50% of the dronabinol in about 10 min to about 30 min using USP Method [symbol] 711 [symbol] with Apparatus 2 (Paddle Apparatus) at 100-150 rpm in water with 10% by mass caprylocaproyl macrogol-8/polyoxyl-8 glycerides (Labrasol®). In another aspect, the when administered to a human subject, has one or more of the follow pharmacokinetic parameters:

| Dose | Cₘₐₓ ng/mL | Tₘₐₓ hr (range) | AUC_{0→12} ng · hr/mL |
|---|---|---|---|
| 2.5 mg | 1.32 (0.62) | 1.00 (0.50-4.00) | 2.88 (1.57) |
| 5 mg | 2.96 (1.81) | 2.50 (0.50-4.00) | 6.16 (1.85) |
| 10 mg | 7.88 (4.54) | 1.50 (0.50-3.50) | 15.2 (5.52) |

Cₘₐₓ: maximum observed plasma concentration;
Tₘₐₓ: Median time to maximum observed plasma concentration;
AUC_{0→12}: area under the plasma concentration-time curve from 0 to 12 hours

Another embodiment described herein is a method for manufacturing the pharmaceutical dosage forms described herein comprising the steps of: preparing a shell composition comprising one or more of gelatin, sorbitol, and water; mixing the shell composition under vacuum for 1 to 2 hours at a temperature of at least about 50° C.; preparing a matrix fill composition comprising combining one or more cannabinoids, gelatins, gelatin hydrolysates, low melting hard fats, optional antioxidants, sorbitol, and water; mixing the matrix fill composition at a temperature of about 40° C. to 50° C.; casting the shell composition of (b) into films or ribbons using heat-controlled drums or surfaces while incorporating the matrix fill composition of (d); and forming a soft dosage form comprising a liquid matrix fill using rotary die encapsulation technology.

Another embodiment described herein is an oral pharmaceutical dosage form comprising a soft capsule dosage form comprising a stable form of dronabinol produced by any of the method described herein.

Another embodiment described herein is a method for treating a subject suffering from or having the symptoms of anorexia associated with weight loss in AIDS by orally administering a pharmaceutical dosage form described herein to the subject in need thereof.

Another embodiment described herein is a method for treating a subject suffering from or having the symptoms of nausea and vomiting associated with cancer chemotherapy by orally administering a pharmaceutical dosage form described herein to the subject in need thereof.

Another embodiment described herein is a method for treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders by orally administering a pharmaceutical dosage form described herein to the subject in need thereof.

Another embodiment described herein is a composition as described herein for use in treating a subject suffering from or having the symptoms of anorexia associated with weight loss in AIDS or nausea and vomiting associated with cancer chemotherapy.

Another embodiment described herein is a composition as described herein for use in treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders.

Another embodiment described herein is a kit for dispensing a pharmaceutical dosage form described herein comprising: (a) at least one dosage form described herein; (b) at least one receptacle comprising a tamper evident, moisture proof packaging comprising blister or strip packs, aluminum blister, transparent or opaque polymer blister with pouch, polypropylene tubes, colored blister materials, tubes, bottles, and bottles optionally containing a child-resistant feature, optionally comprising a desiccant, such as a molecular sieve or silica gel, an inert atmosphere such as nitrogen or helium; and an antioxidant or oxygen absorber; and (c) optionally, an insert comprising, a label, instructions, or prescribing information.

### DETAILED DESCRIPTION

One embodiment described herein is a stable pharmaceutical composition comprising a cannabinoid. In one aspect, the cannabinoid is dronabinol. In another aspect, the composition is a soft gelatin capsule encapsulating a liquid or semi-solid composition that stabilizes dronabinol and does not require refrigeration.

As used herein, the term “dronabinol” refers to the cannabinoid Δ⁹-tetrahydrocannabinol having the CAS number 1972-08-03, the IUPAC name, (6aR,10aR)-6,6,9-trimethyl-3-pentyl-6a,7,8,10a-tetrahydro-6H-benzo[c]chromen-1-ol, and the following chemical structure (1):

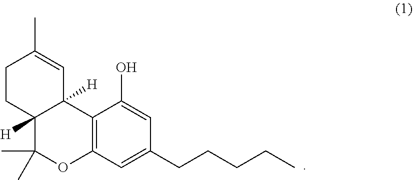 [SMILES: CCCCCc1cc(O)c2c(c1)OC(C)(C)[C@@H]1CCC(C)=C[C@@H]21]

The formulations and methods described herein provide stable pharmaceutical compositions of any cannabinoid.

As used herein, the term “cannabinoids” refer to the phytocannabinoids from Cannabis. The compounds may be synthetic or natural products isolated from or extracted from Cannabis sativa, Cannabis indica, or Cannabis ruderalis. Exemplary cannabinoids include (−)-Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); Δ⁹-tetrahydrocannabivarin (Δ⁹-THCBV); Δ⁸-tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-NE); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); Δ⁹-tetrahydrocannabibutoic acid (Δ⁹-THCBA); Δ⁸-tetrahydrocannabibutolic acid (Δ⁸-THCBA); Δ⁹-tetrahydrocannabibutol naphtoylester (Δ⁹-THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof. See e.g., Burstein et al., J. Med. Chem. 35(17): 3135-3141 (1992). “Cannabinoids” include isolated natural compounds, synthetic, and semi-synthetic cannabinoids. Semi-synthetic cannabinoids include non-natural derivatives of cannabinoids that can be obtained by derivatization of natural cannabinoids. Synthetic or purified cannabinoids are available from Purisys and Cayman Chemical. See Ultra-Pure Cannabinoids, Purisys™ Advanced Cannabinoids, Athens, Ga. (2019) and Cayman Currents, Phytocannabinoids: what is on the horizon? Issue 34; Cayman Chemical, Ann Arbor Mich. (Fall 2020) both of which are incorporated by reference herein for their specific teachings. The cannabinoid may be included in its free form or in the following forms: a salt; an acid addition salt of an ester; an amide; an enantiomer; an isomer; a tautomer; a prodrug; a derivative of an active agent of the present invention; different isomeric forms, including, but not limited to enantiomers and diastereoisomers, both in pure form and in admixture, including racemic mixtures; and enols. “Cannabinoid” also encompasses derivatives that are produced from compounds having similar structures by the replacement or removal of one atom, molecule, or group by another.

In one embodiment, the cannabinoid comprises a cannabinoid compound that can be isolated from Cannabis, derived from a natural cannabinoid (semi-synthetic), or chemically synthesized. In one embodiment, the cannabinoid comprises a compound having the structure of:

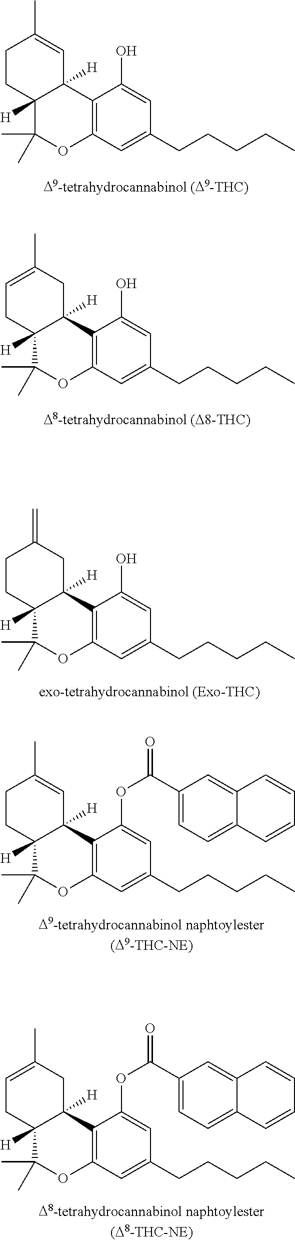

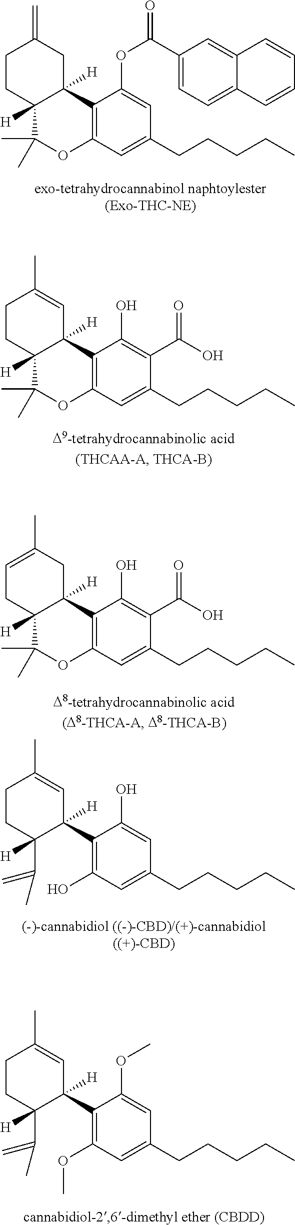

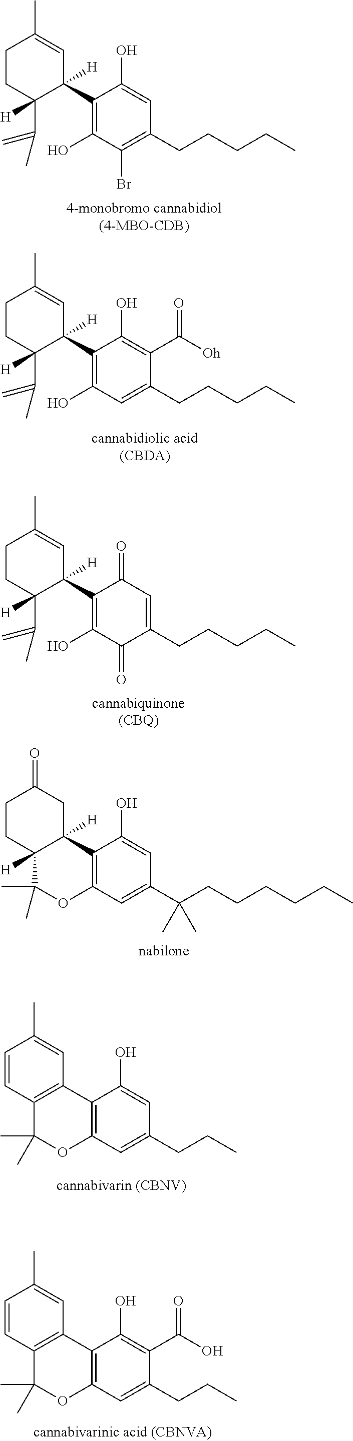

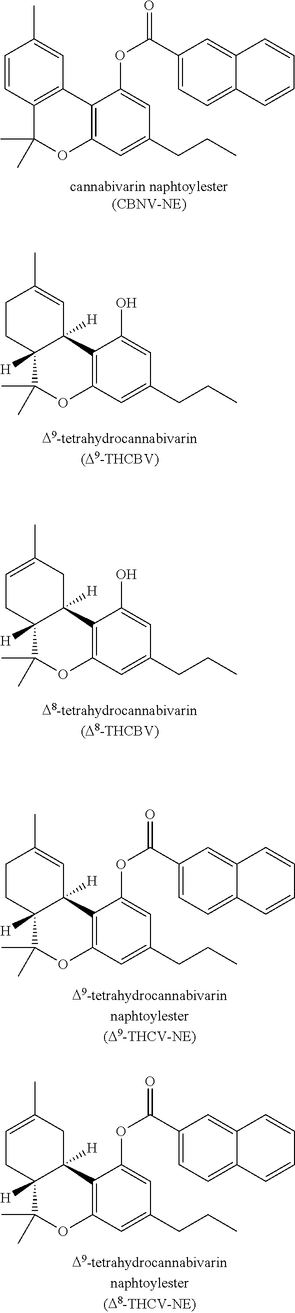

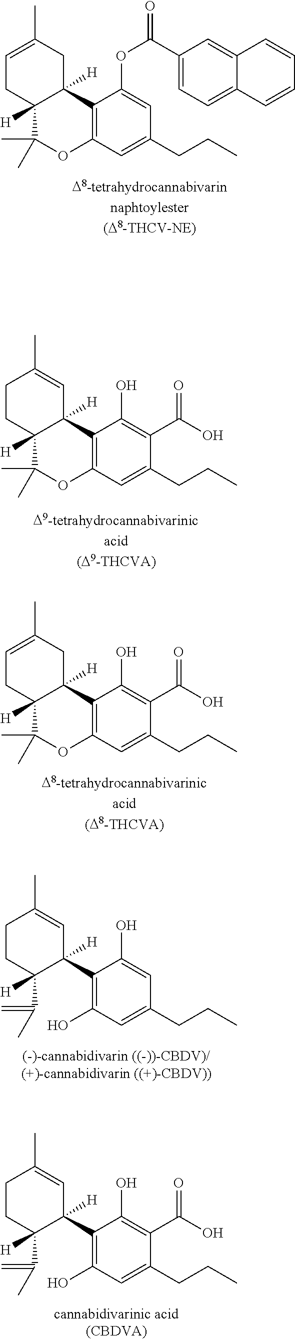

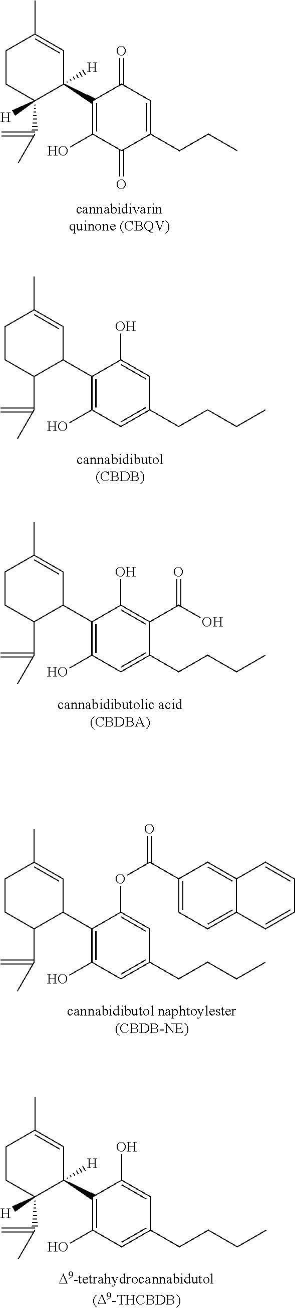

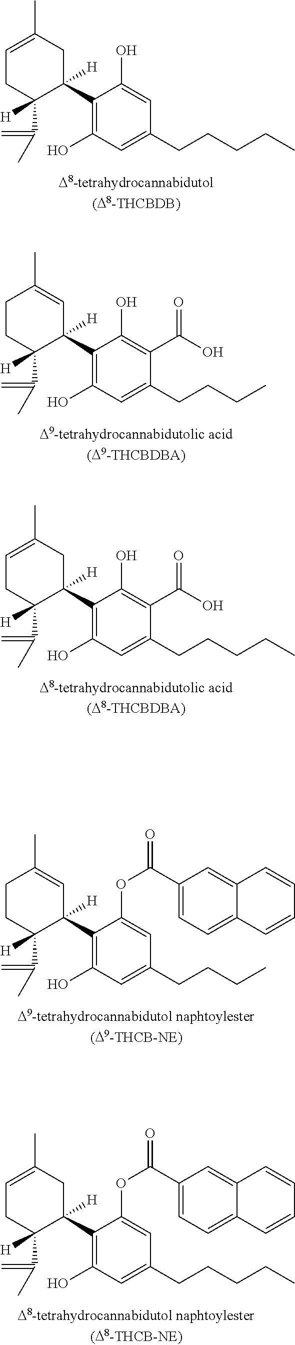

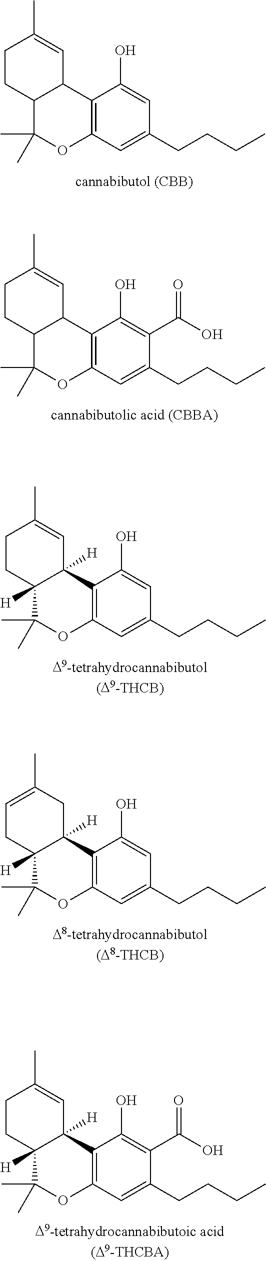

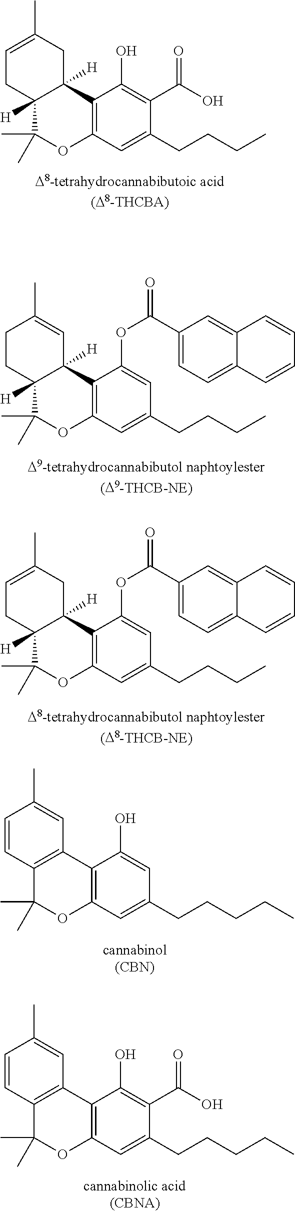

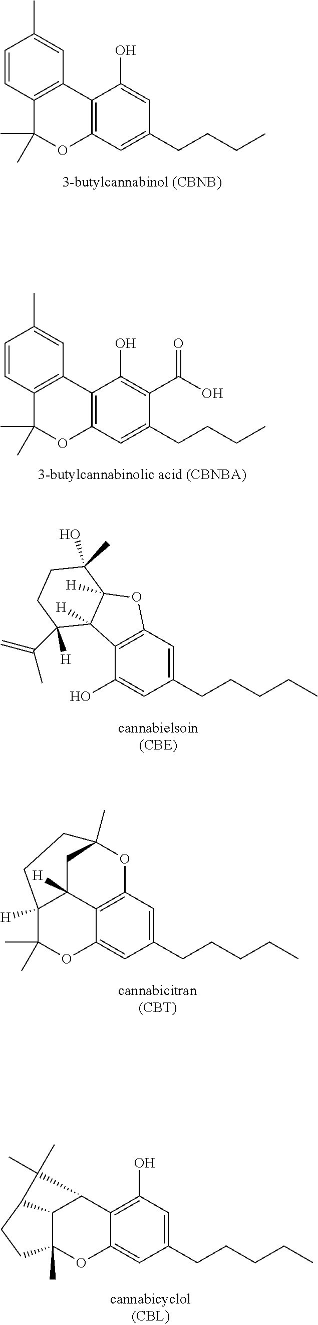

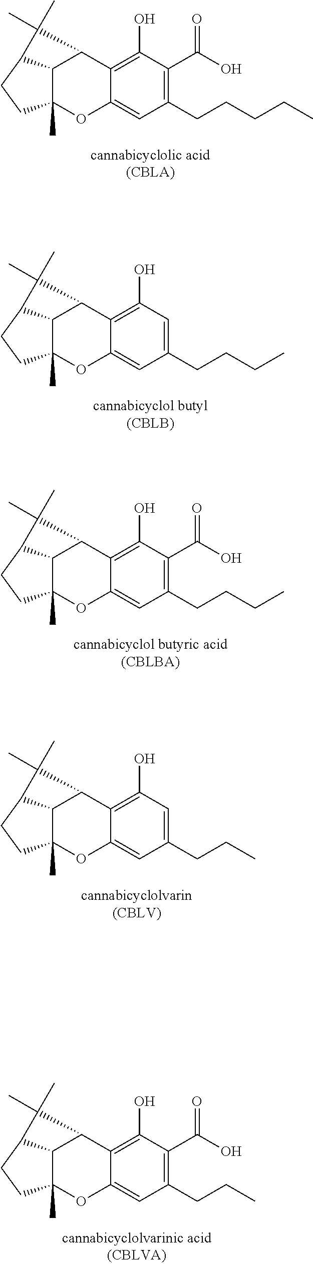

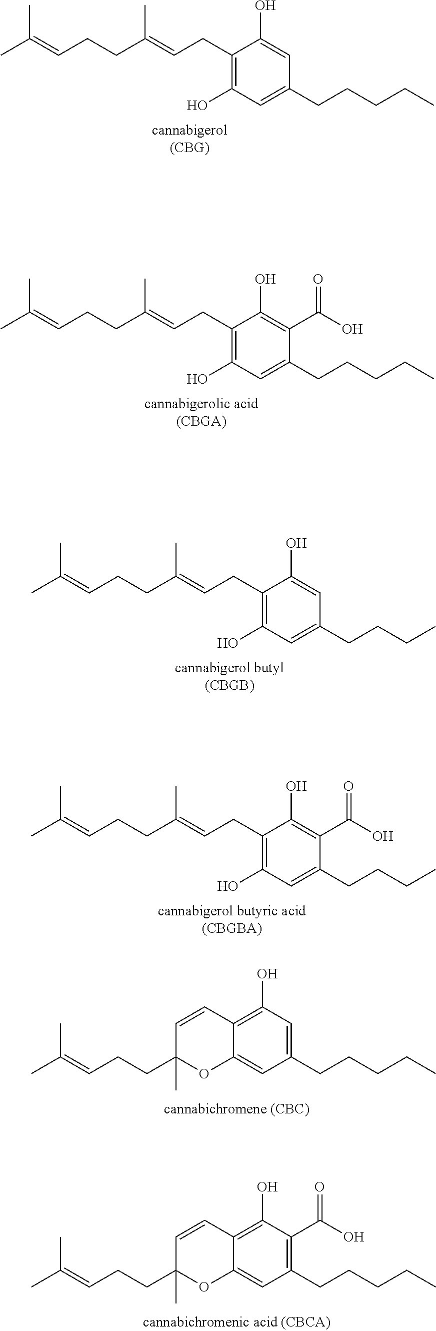

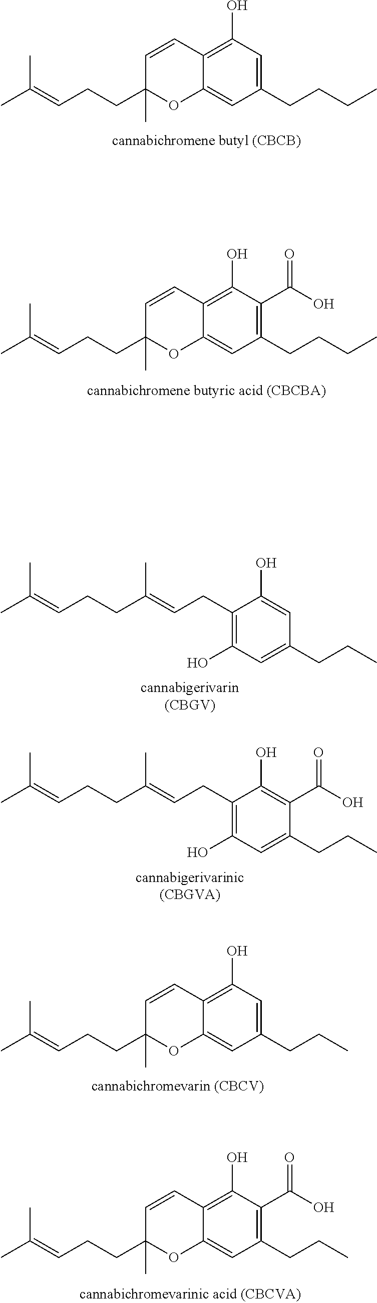

In one embodiment, the cannabinoid is Δ⁹-tetrahydrocannabinol (THC) or dronabinol.

A possible mechanism for the oxidative degradation of Δ⁹-tetrahydrocannabinol to cannabinol (CBN) is shown in Scheme I:

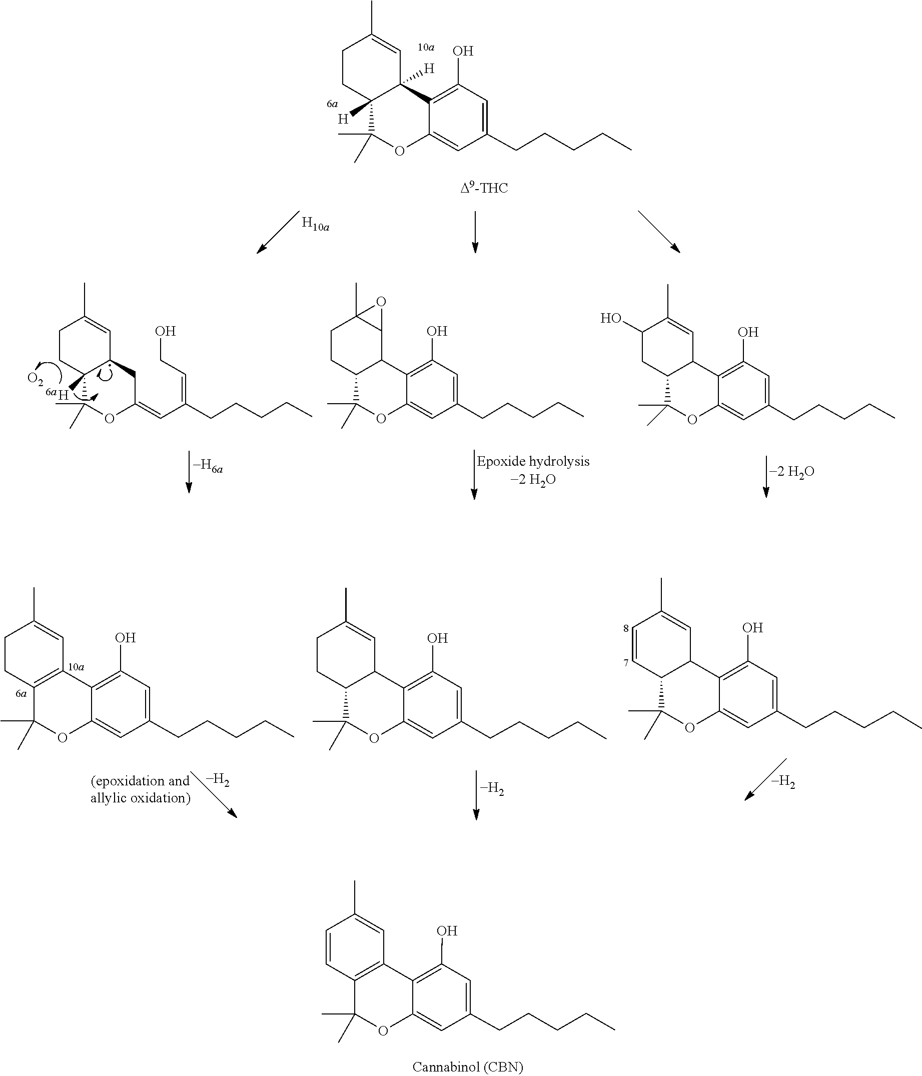

See Hanuš, et al., Nat. Prod. Rep. 33: 1357-1392 (2016), which is incorporated by reference herein for such teachings. Some of the Δ⁹-THC oxidative intermediates shown are also believed to be converted to cannabidiol (CBD). Id.

The term “a therapeutically effective amount” of a compound described herein refers to an amount of the compound described herein that will elicit the biological or medical response of a subject, for example, reduction or inhibition of an enzyme or a protein activity, or ameliorate symptoms, alleviate conditions, slow or delay disease progression, or prevent a disease, etc. In one embodiment, the term “a therapeutically effective amount” refers to the amount of the compound described herein that, when administered to a subject, is effective to at least partially alleviate, prevent or ameliorate a condition, or a disorder. In one embodiment, the term “a therapeutically effective amount” refers to the amount of the compound described herein that, when administered to a cell, or a tissue, or a non-cellular biological material, a medium, or a living subject, including humans, is effective to treat or ameliorate anorexia, lack of appetite, nausea, vomiting, or other gastrointestinal or inflammatory disorders, including chronic or acute pain, trichotillomania, Tourette's syndrome, lower back pain, neuropathic pain, insomnia, cervical dystonia, post-traumatic stress disorder or syndrome (PTSD), migraine headaches, dementia, multiple sclerosis, fibromyalgia, inter alia.

As used herein, the term “subject” refers to an animal. Typically, the animal is a mammal. A subject refers to, for example, primates (e.g., humans, male or female; infant, adolescent, or adult), cows, sheep, goats, horses, dogs, cats, rabbits, rats, mice, fish, birds, and the like. In one embodiment, the subject is a primate. In one embodiment, the subject is a human. In one aspect, the subject is a chemotherapy patient. In another aspect, the subject is an AIDS patient. In another aspect, the subject is a human experiencing lack of appetite, nausea, vomiting, or other gastrointestinal disorder. In another aspect the subject is a human experience chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders.

As used herein, the terms “inhibit,” “inhibition,” or “inhibiting” refer to the reduction or suppression of a given condition, symptom, or disorder, or disease, or a significant decrease in the baseline activity of a biological activity or process.

The term “soft capsule” or “soft gel capsule” as used herein refers to a soft capsule comprising one or more film-forming polymers that is capable of encapsulating a liquid “matrix” or “fill” comprising pharmaceutically acceptable excipients and one or more active pharmaceutical ingredients.

The terms “matrix,” “fill,” or “matrix fill” as used herein refer to a composition comprising one or more active pharmaceutical ingredients that is encapsulated within a capsule. Often the matrix comprises a vehicle, one or more active pharmaceutical ingredients, and one or more pharmaceutically acceptable excipients. In one aspect described herein, the matrix is a liquid or a semisolid, comprises a lipid or lipophilic composition comprising one or more cannabinoids.

The terms “active pharmaceutical ingredient load” or “drug load” as used herein refers to the quantity (mass) of the active pharmaceutical ingredient comprised in a single soft capsule fill.

The terms “formulation” or “composition” as used herein refers to the drug in combination with pharmaceutically acceptable excipients. This term includes orally administrable formulations as well as formulations administrable by other means.

The term “titration” as used herein refers to the incremental increase in drug dosage to a level that provides the optimal therapeutic effect.

The term “controlled release” as used herein encompasses the terms “immediate release,” “modified release,” “sustained release,” “extended release,” and “delayed release.”

The terms “extended release” or “sustained release” as used herein refers to a composition that releases an active ingredient according to a desired profile over an extended period under physiological conditions or in an in vitro test. By “extended period” it is meant a continuous period of time of at least about 1 hour; about 2 hours; about 4 hours; about 6 hours; about 8 hours; about 10 hours; about 12 hours; about 14 hours; about 16 hours; about 18 hours; about 20 hours about 24 hours; or even longer; specifically, over a period of about 18 hours under physiological conditions or in an in vitro assay.

The term “modified release” as used herein refers to a composition that releases an active ingredient at a slower rate than does an immediate release formulation under physiological conditions or in an in vitro test.

The term “delayed” release” as used herein refers to a composition that releases an active ingredient after a period of time, for example minutes or hours, such that the active ingredient is not released initially. A delayed release composition may provide, for example, the release of a drug or active ingredient from a dosage form, after a certain period, under physiological conditions or in an in vitro test.

The term “Cₘₐₓ” as used herein refers to the maximum observed blood (plasma, serum, or whole blood) concentration or the maximum blood concentration calculated or estimated from a concentration to time curve, and is expressed in units of mg/L or ng/mL, as applicable.

The term “Cₘᵢₙ” as used herein refers to the minimum observed blood (plasma, serum, or whole blood) concentration or the minimum blood concentration calculated or estimated from a concentration to time curve, and is expressed in units of mg/L or ng/mL, as applicable.

The term “C_{avg}” as used herein refers to the blood (plasma, serum, or whole blood) concentration of the drug within the dosing interval, is calculated as AUC/dosing interval, and is expressed in units of mg/L or ng/mL, as applicable.

The term “Tₘₐₓ” as used herein refers to the time after administration at which Cₘₐₓ occurs and is expressed in units of hours (h) or minutes (min), as applicable.

The term “AUC_{0→T}” as used herein refers to area under the blood (plasma, serum, or whole blood) concentration versus time curve from time zero to time tau (i) over a dosing interval at steady state, where tau is the length of the dosing interval, and is expressed in units of h·mg/L or h·ng/mL, as applicable. For example, the term AUC_{0→12} as used herein refers to the area under the concentration versus time curve from 0 to 12 hours.

The term “AUC_{0→∞}” as used herein refers to the area under the blood (plasma, serum, or whole blood) concentration versus time curve from time 0 hours to infinity, and is expressed in units of h·mg/L or h·ng/mL, as applicable.

The terms “bioequivalence” or “bioequivalent” as used herein refer to a drug product or dosage form that has highly similar release and systemic absorption as compared to a reference drug. The U.S. Food, Drug and Cosmetic Act (21 U.S.C. § 505(j)(8)(B)(i)) provides that a drug is bioequivalent to a reference listed drug (RLD) if: “the rate and extent of absorption of the drug do not show a significant difference from the rate and extent of absorption of the listed drug when administered at the same molar dose of the therapeutic ingredient under similar experimental conditions in either a single dose or multiple doses . . . .”

The phrase “enhanced bioavailability” as used herein refers to the increased proportion of an active pharmaceutical ingredient that enters the systemic circulation when introduced into the body as compared to a reference active pharmaceutical's bioavailability. Bioavailability can be determined by comparing the rate and extent of absorption of a test drug with a reference drug when administered at the same molar dose of the active therapeutic ingredient under similar experimental conditions in either a single dose or multiple doses. Typical pharmacokinetic parameters can be used to demonstrate enhanced bioavailability compared to the reference.

The phrase “enhanced stability,” “stable,” or “enhanced oxidative stability” as used herein refers to the increased stability of a compound after isolation or synthesis, during manufacturing, or in its final dosage form. “Enhanced stability” refers to the ability of the compound to resist oxidation, reduction, or degradation and remain in a substantially chemically pure form. Most cannabinoids are highly susceptible to oxidation. Current formulations of dronabinol require storage in light-proof containers and storage under refrigerated (2-8° C.) or cool (8-15° C.) conditions. The compositions and formulations described herein have enhanced stability compared to the current formulations because they are less susceptible to oxidation and do not require refrigeration for stability (although their stability can be further enhanced by cold storage).

As used herein, the terms “treat”, “treating,” or “treatment” of any disease or disorder refer In an embodiment, to ameliorating the disease or disorder (i.e., slowing or arresting or reducing the development of the disease or at least one of the clinical symptoms thereof). In an embodiment, “treat,” “treating,” or “treatment” refers to alleviating or ameliorating at least one physical parameter including those which may not be discernible by the patient.

As used herein, the term “preventing” refers to reducing the incidence of, reducing the frequency of, or delaying the onset of, symptoms or conditions of a disease or disorder.

The term “prophylaxis” refers to preventing, reducing the incidence of, reducing the frequency of, delaying the onset of, or halting or reducing the progression of a disease or disorder, either to a statistically significant degree or to a degree detectable to one skilled in the art.

As used herein, a subject is “in need of” a treatment if such subject would benefit biologically, medically, or in quality of life from a treatment as described herein.

The term “substantially” as used herein means to a great or significant extent, but not completely.

As used herein, ambient temperature refers to temperatures typical of a controlled environment. In one aspect ambident temperature is “room temperature” which include temperatures of about 20° C. to about 30° C., including each integer and end point within the specified range. In one aspect, ambient temperature is 25±1° C.; 25±2° C.; 25±3° C.; 25±4° C.; or 25±5° C. In another aspect, ambient temperature is 25±2° C.

As used herein, all percentages (%) refer to mass (or weight, w/w) percent unless noted otherwise.

The term “about” as used herein refers to any values, including both integers and fractional components that are within a variation of up to ±10% of the value modified by the term “about.”

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

Terms such as “include,” “including,” “contain,” “containing,” “having,” and the like mean “comprising.”

The term “or” can be conjunctive or disjunctive.

Certain compounds described herein may exist in particular geometric or stereoisomeric forms. A particular enantiomer of a compound described herein may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers.

Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer. ee=(90−10)/100×100=80%.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words, such compounds or compositions contain an enantiomeric excess of the S enantiomer over the R enantiomer.

When a particular enantiomer is preferred, it may be provided substantially free of the corresponding alternative enantiomer and may also be referred to as “optically enriched.” “Optically enriched,” as used herein, means that the compound is made up of a significantly greater proportion of one enantiomer. In certain embodiments, the compound is made up of at least about 90% by mass of a preferred enantiomer. In other embodiments, the compound is made up of at least about 95%, 98%, or 99% by mass of a preferred enantiomer. Preferred enantiomers may be isolated from racemic mixtures by any method known to those skilled in the art, including chiral high-pressure liquid chromatography (HPLC) and the formation and crystallization of chiral salts or prepared by asymmetric syntheses. See e.g., Jacques et al., Enantiomers, Racemates and Resolutions, Wiley Interscience, New York (1981); Wilen, et al., Tetrahedron 33:2725 (1977); Eliel, E. L. Stereochemistry of Carbon Compounds, McGraw Hill, N Y (1962); Wilen, S. H. Tables of Resolving Agents and Optical Resolutions p. 268, E. L. Eliel, Ed., Univ. of Notre Dame Press, Notre Dame, Ind. (1972).

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

Any resulting mixtures of isomers can be separated based on the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Various embodiments of the disclosure are described herein. It will be recognized that features specified in each embodiment may be combined with other specified features, including as indicated in the embodiments below, to provide further embodiments of the present disclosure.

### Pharmaceutically Acceptable Salts

Pharmaceutically acceptable salts of the cannabinoid compounds described herein are also contemplated for the uses described herein. As used herein, the terms “salt” or “salts” refer to an acid addition or base addition salt of a compound described herein. “Salts” include in particular “pharmaceutical acceptable salts.” The term “pharmaceutically acceptable salts” refers to salts that retain the biological effectiveness and properties of the compounds disclosed herein and, which typically are not biologically or otherwise undesirable. In many cases, the compounds disclosed herein are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine, and tromethamine.

### Pharmaceutical Compositions

One embodiment described is a pharmaceutical composition comprising one or more cannabinoid compounds described herein or a pharmaceutically acceptable salt, hydrate, solvate, prodrug, stereoisomer, or tautomer thereof, and one or more pharmaceutically acceptable carrier(s). The term “pharmaceutically acceptable carrier” refers to a pharmaceutically-acceptable material, composition, or vehicle, such as a liquid or solid filler, diluent, excipient, solvent, or encapsulating material, involved in carrying or transporting any subject composition or component thereof. Each carrier must be “acceptable” in the sense of being compatible with the subject composition and its components and not injurious to the patient. In one embodiment, the pharmaceutical composition comprises one or more cannabinoids. In one aspect, the pharmaceutical composition comprises Δ⁹-tetrahydrocannabinol (Δ⁹-THC or THC), also known as dronabinol. In one embodiment, the pharmaceutical composition comprises a soft gelatin capsule (“softgel”) comprising dronabinol and one or more pharmaceutically acceptable excipients that stabilize the dronabinol and prevent oxidation, reduction, esterification, hydrolysis, or other forms of chemical modification or degradation at room temperature.

In one embodiment, the pharmaceutical composition comprises a soft gelatin capsule encapsulating a “matrix” or “fill” comprising a cannabinoid.

In another embodiment, the composition comprises a lipid or lipophilic vehicle that provides a liquid, semi-solid, or solid suspension of a cannabinoid. In one aspect, a capsule comprising a lipid or lipophilic vehicle comprising semi-solid, or solid suspension of one or more cannabinoids provides controlled release delivery of the cannabinoid. In one embodiment, the capsule is a soft capsule. In another embodiment, the capsule is a hard capsule. In another embodiment, the capsule is coated with one or more subcoatings, one or more enteric coatings, and one or more topcoating moisture barriers.

In another embodiment, the pharmaceutical composition comprises softgel fills for cannabinoids, prodrugs thereof, or derivatives thereof, based on lipid or lipophilic vehicles. Some of the described fill composition have a hydrophobic (lipophilic) surface in contact with the hydrophilic soft capsule shell to minimize any potential shell-fill interactions, such as when soft capsules are filled with hydrophilic vehicles.

Described herein are methods for manufacturing composition comprising cannabinoids, prodrugs thereof, or derivatives thereof, in a controlled release soft capsule in the form of a suspension, where part or all of the cannabinoid is suspended within the composition. Also provided are compositions and formulations where the cannabinoid is incorporated into a single-phase or two-phase composition or an emulsion.

Also described herein are methods for manufacturing compositions comprising cannabinoid or derivatives thereof, in a delayed release soft capsule in the form of a suspension, where part or all of the cannabinoid is suspended within the composition.

Described herein are methods for manufacturing compositions comprising cannabinoid or derivatives thereof, in an extended release soft capsule in the form of a suspension, where part or all of the cannabinoid is suspended within the composition.

Another embodiment described herein is a controlled, delayed, or extended release dosage form comprising a soft gelatin capsule having a shell and a matrix fill, wherein the matrix fill includes a lipid or lipophilic liquid vehicle comprising a cannabinoid. In one aspect, the matrix fill further comprises a solid or semi-solid lipid. In another aspect, the matrix fill further comprises one or more antioxidants. In another aspect, the matrix fill further comprises an emulsifier. In other aspects, the matrix fill further comprises a solvent.

In one aspect, the composition comprises the composition shown in Table 1:

TABLE 1
Exemplary Composition

| | | Mass |
|---|---|---|
| Ingredient | Mass (mg) | percent (%) |
| Cannabinoid (e.g., dronabinol) in vehicle | 2.5, 5, or 10 | 1-10% |
| (e.g., sesame oil or EtOH) | | |
| Vehicle (sesame oil, vegetable oil, or | 0-170 | 40-85% |
| other pharmaceutically acceptable | | |
| lipid or lipophilic vehicles) | | |
| Optional low-melting hard lipid (e.g., | 0-100 | 0-50% |
| Gelucire ® 43/01 and/or Gelucire ® | | |
| 50/13) | | |
| Optional antioxidant(s) (e.g., propylene | 0-40 | 0-20% |
| gallate, BHT, tocopherol, D/L-α- | | |
| tocopherol, Vitamin E, Vitamin E-TPGS, | | |
| lecithin, etc.) | | |
| TOTAL | ~150-200 mg | 100% |

In one embodiment, the composition comprises a lipid or lipophilic vehicle comprising a liquid lipophilic vehicle, a semisolid lipophilic vehicle, or a mixture thereof. Suitable lipid or lipophilic vehicles include mineral oil; light mineral oil; natural oils (e.g., vegetable, corn, canola, sunflower, soybean, olive, coconut, cocoa, peanut, almond, cottonseed, persic, sesame, squalane, castor, cod liver, etc) hydrogenated vegetable oil; partially hydrogenated oils; bee's wax (beeswax); polyethoxylated bee's wax; paraffin; normal waxes; medium chain medium chain monoglycerides, diglycerides and triglycerides; higher aliphatic alcohols; higher aliphatic acids; long chain fatty acids; saturated or unsaturated fatty acids; hydrogenated fatty acids; fatty acid glycerides; polyoxyethylated oleic glycerides; monoglycerides and diglycerides; mono-, bi- or tri-substituted glycerides; glycerol mono-oleate esters; glycerol mono-caprate; glyceryl monocaprylate; propylene glycol dicaprylate; propylene glycol monolaurate; glyceryl palmitostearate; glyceryl behenate; diethyleneglycol palmitostearate; polyethyleneglycol stearate; polyoxyethyleneglycol palmitostearate; glyceryl mono palmitostearate; cetyl palmitate; polyethyleneglycol palmitostearate; dimethylpolysiloxane; mono- or di-glyceryl behenate; fatty alcohols associated with polyethoxylate fatty alcohols; cetyl alcohol; octyldodecanol; myristyl alcohol; isopropyl myristate, isopropyl palmitate, stearic acid, stearyl alcohol, and others known in the art. In one aspect, the lipid or lipophilic vehicle or solvent is sesame oil.

In another embodiment, the composition comprises a liquid lipophilic vehicle and a solid or semisolid lipophilic vehicle, such as a low melting hard lipid. In one embodiment, the liquid lipid or lipophilic vehicle can be sesame oil, olive oil, sunflower oil, canola oil, palmitoleic acid, oleic acid, myristoleic acid, linoleic acid, arachidonic acid, paraffin oil, mineral oil, or propylene glycol dicaprylate/dicaprate (Miglyol® 840). In one aspect, the liquid lipid vehicle is sesame oil. In one aspect, the liquid lipid vehicle is or propylene glycol dicaprylate/dicaprate (Miglyol® 840).

In another embodiment, the composition comprises one or more low melting hard lipids such as polyethylene glycol glyceride esters, paraffin wax, or bee's wax. In another embodiment, the low melting hard lipid is a polyethylene glycol glyceride. In one aspect, the polyethylene glycol glyceride comprises one or more polyethylene glycol (PEG) mono-, di-, or tri-glyceride fatty acid esters (C8 to C18). In one aspect, the polyethylene glycol glyceride comprises: Gelucire® 33/01, Gelucire® 37/02, Gelucire® 39/01, Gelucire® 43/01, Gelucire® 44/14, Gelucire® 50/02, Gelucire® 50/13, Gelucire® 53/10, or Gelucire® 62/02 (Gattefossé). In another aspect, the low melting hard lipid is a comprises a polyethylene glycol glyceride, e.g., Gelucire®. In one aspect, the polyethylene glycol glyceride is a Gelucire® semisolid lipid with an HLB value of about 1 and a melting point of about 42° C. to about 46° C. In one aspect, the low melting hard lipid is Gelucire® 43/01. the low melting hard lipid is a comprises a polyethylene glycol glyceride, e.g., Gelucire®. In one aspect, the polyethylene glycol glyceride is a Gelucire® semisolid lipid with an HLB value of about 13 and a melting point of about 50° C. to about 56° C. In one aspect, the low melting hard lipid is Gelucire® 53/13. In one aspect, the low melting hard lipid is one or more of Gelucire® 43/01 or Gelucire® 53/13. In one aspect, the low melting hard lipid is Gelucire® 43/01, a polyethylene glycol glyceride with a melting point of 43° C. and an HLB of about 1. In one aspect, the low melting hard lipid is Gelucire® 53/13, a polyethylene glycol glyceride with a melting point of 53° C. and an HLB of about 13.

In another embodiment, the composition comprises one or more antioxidants. The composition can comprise butylated hydroxyanisole (BHA), butylated hydroxytoluene (BHT), propylene gallate, lecithin, vitamin A tocopherol, D/L-α-tocopherol, vitamin E tocopherol, vitamin E polyethylene glycol succinate (TPGS), sesamin, sesamol, sesamolin, ascorbic acid, ascorbyl palmitate, sodium ascorbate, fumaric acid, malic acid, sodium metabisulphite, disodium ethylenediaminetetracetic acid (EDTA), or combinations thereof. In one aspect, the antioxidant comprises one or more of butylated hydroxyanisole (BHA), butylated hydroxytoluene (BHT), sesamin, sesamol, sesamolin, or combinations thereof.

In another embodiment, the composition comprises one or more gelling agents. In one aspect, the gelling agent comprises gelatin or a gelatin hydrolysate. In one aspect, the gelling agent comprises gelatin, gelatin hydrolysate, a carrageenan, methylcellulose, hydroxypropyl methylcellulose, starch, polyacrylates, polyvinyl polymers, or a combination thereof. In one aspect, the gelling agent comprises gelatin having a Bloom value of 50 to 100, a gelatin hydrolysate, or a combination thereof. In one aspect, the gelling agent comprises gelatin having a Bloom value of 50 to 100 and a gelatin hydrolysate.

In one embodiment, the composition comprises a solvent or solubility enhancing agent. Exemplary solvents or solubility enhancing agents useful for the compositions described herein include Capmul® MCM, Cremophor® RH 40, Captex® 355, Croscarmellose, Crospovidone, Crospovidone CL, Crospovidone CL-F, Crospovidone CL-M, Imwitor® 742, Kollidon® CL, Kollidon® CL-F, Kollidon® CL-M, Labrafac™ Lipophile WL 1349, Labrafil® M2125CS, Labrasol®, Lutrol® F 68, Maisine™ 35-1, mannitol, Miglyol® 812, Miglyol® 840®, Pearlitol® Flash, Peceol®, polyethylene glycol 400, polyethylene glycol 600, polyethylene glycol 3350, Plurol® Oleique CC 497, Povidone K 17, Povidone K 30, propylene glycol, or combinations thereof. In one embodiment, the solvent is propylene glycol dicaprylate/dicaprate or Miglyol® 840.

In another embodiment, the composition comprises one or more hydrophilic solvents or suspension agents. The composition can comprise polyvinylpyrrolidone, polyethylene glycols of molecular weight ranging from about 200 to about 8000 (M_{N}, number average molecular weight), or combinations thereof.

In one embodiment, the composition comprises a hydrophilic ionic polymer. In one embodiment, the hydrophilic polymers comprise polyhydroxylalkylenediamine, dimethylaminoethyl methacrylate copolymer, Poly(butyl methacrylate-co-(2-dimethylaminoethyl) methacrylate-co-(2-dimethylaminoethyl) 1:2:1 (Eudragit® EPO); sodium carboxy methylcellulose, carboxymethyl cellulose ethylenediamine, sodium alginate, alginic acid, pectin, carbomers, Carbopol® copolymers (polyacrylic acid polymers), such as Carbopol® 934, Carbopol® 940, Carbopol® 941 or Carbopol® 974P; a Pemulen® polymer; polycarbophil poly galacturonic acid, polyglucoronic acid, chondroitic sulfate, carrageenan, and acrylic methacrylate copolymers. In one aspect, the hydrophilic polymer swells in aqueous media.

In another embodiment, the composition comprises a hygroscopic polymer. In one embodiment, the hygroscopic polymers include polyvinylpyrrolidone, copovidone, hydroxypropylmethylcellulose, hydroxypropylcellulose, ethyl cellulose, methylcellulose, and polyethylene oxide. Suitable hygroscopic polymers include polyvinyl alcohol, a copolymer of polyvinylpyrrolidone and polyvinyl acetate, hydroxypropyl cellulose, hydroxypropyl methylcellulose, hydroxyethyl cellulose, hydroxymethyl cellulose, gelatins, polyethylene oxide, such as POLYOX™ 100,000-600,000 MW, acacia, dextrin, starch, polyhydroxyethylmethacrylate, a water-soluble non-ionic polymethacrylate or copolymer thereof, a modified cellulose, a modified polysaccharide, a non-ionic gum, or a non-ionic polysaccharide.

In another embodiment, the composition comprises one or more release regulator such as a fatty acid salt, fatty acid ester, or fatty acid polyoxyethylene derivative. The release regulator can also be a surfactant having a hydrophilic/lipophilic balance (HLB) value between about 2 and about 40. The HLB characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993), which is incorporated by reference herein for such teachings.

In another embodiment, the composition comprises one or more emulsifiers or solubilizing agents such as acacia, cholesterol, diethanolamine, glyceryl monostearate, lanolin alcohols, lecithin, mono- and di-glycerides, monoethanolamines, oleic acids, oleyl alcohols, poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monostearate, stearic acid, trolamine, emulsifying wax, or combinations thereof.

In one embodiment, the composition comprises a non-ionic surfactant. The surfactant can have a hydrophilic/lipophilic balance (HLB) value between about 1 and about 25 and a melting point between about 25° C. and about 70° C. The HLB characteristic of surfactants can be determined in accordance with “Physical Pharmacy: Physical Chemical Principles in the Pharmaceutical Sciences,” Fourth Edition, pp. 371-373, A. Martin, Ed., Lippincott Williams & Wilkins, Philadelphia (1993). Suitable non-ionic surfactants include: Pluronic® 10R5, Pluronic® 17R2, Pluronic® 17R4, Pluronic® 25R2, Pluronic® 25R4, Pluronic® 31R1, Pluronic® F 108, Pluronic® F 108 NF, Pluronic® F 108, Pluronic® F 108NF, Poloxamer 338, Pluronic® F 127, Pluronic® F 127 NF, Pluronic® F 127 NF 500 BHT Prill, Pluronic® F 127 NF Prill, Poloxamer 407, Pluronic® F 38, Pluronic® F 38 Pastille, Pluronic® F 68, Pluronic® F 68 LF Pastille, Pluronic® F 68 NF, Pluronic® F 68 NF Prill, Poloxamer 188, Pluronic® F 68 Pastille, Pluronic® F 77, Pluronic® F 77 Micropastille, Pluronic® F 87, Pluronic® F 87 NF, Pluronic® F 87 NF Prill, Poloxamer 237, Pluronic® F 88, Pluronic® F 88 Pastille, Pluronic® F 98, Pluronic® L 10, Pluronic® L 101, Pluronic® L 121, Pluronic® L 31, Pluronic® L 35, Pluronic® L 43, Pluronic® L 61, Pluronic® L 62, Pluronic® L 62 LF, Pluronic® L 62D, Pluronic® L 64, Pluronic® L 81, Pluronic® L 92, Pluronic® N 3, Pluronic® P 103, Pluronic® P 104, Pluronic® P 105, Pluronic® P 123 Surfactant, Pluronic® P 65, Pluronic® P 84, Pluronic® P 85, Adogen® 464, Alkanol® 6112, Brij® 52, Brij® 93, Brij® S2, Brij® S, Brij® 58, Brij® C10, Brij® L4, Brij® 010, Brij® 010, BRIJ® 020, Brij® S10, Brij® S20, ethylenediamine tetrakis(ethoxylate-block-propoxylate) tetrol, ethylenediamine tetrakis(ethoxylate-block-propoxylate) tetrol, ethylenediamine tetrakis(propoxylate-block-ethoxylate) tetrol, IGEPAL® CA-210, IGEPAL® CA-520, IGEPAL® CA-720, IGEPAL® CO-520, IGEPAL® CO-630, IGEPAL® CO-720, IGEPAL® CO-890, IGEPAL® DM-970, MERPOL® DA, MERPOL® HCS, MERPOL® OJ, MERPOL® SE, MERPOL® SH, MERPOL® A, Poly(ethylene glycol) sorbitan tetraoleate, poly(ethylene glycol) sorbitol hexaoleate, poly(ethylene glycol) (12), poly(ethylene glycol) (18), polyethylene-block-poly(ethylene glycol), sorbitan monopalmitate, 2,4,7,9-tetramethyl-5-decyne-4,7-diol ethoxylate, Nonidet™ P-40, Triton™ N-101, Triton™ X-100, Triton™ X-114, Triton™ X-405, TWEEN® 20, TWEEN® 40, TWEEN® 60, TWEEN® 85, Zonyl® FS-300, or Zonyl® FSN.

In another embodiment, the composition comprises a pH buffering agent. Suitable pharmaceutically acceptable buffering agents comprise arginine, aminomethyl propanol, tetrahydroxypropyl ethylenediamine, triethanolamine, tromethamine, PEG-15 cocamine, di-isopropanol amine, tri-isopropanol amine, N-methyl-D-glucamine, glycine, malate, tartarate, lactate, citrate, acetate, sodium bicarbonate, sodium phosphate, or other buffering agents, having pKₐs at any physiologically acceptable pH, generally from about pH 4 to about pH 7. Amino acids or other physiological metabolites may be used as buffering agents. A combination of buffering agents may also be employed, such as phosphate and acetate, and the like.

In another embodiment, the composition comprises an acidic or basic neutralizing agent. Suitable pharmaceutically acceptable neutralizing agents comprise HCl, phosphoric acid, carbonic acid, sodium hydroxide, ammonium hydroxide, potassium hydroxide, sodium bicarbonate, sodium carbonate, and the like.

In another embodiment, the composition comprises a suspension agent. Suitable pharmaceutically acceptable suspension agents comprise fumed silicon dioxide, such as Aerosil® 90, Aerosil® 130, Aerosil® 150, Aerosil® 200, Aerosil® 255, Aerosil® 300, Aerosil® 380, Aerosil® OX 50, Aerosil® TT 600, Aerosil® 200 F, Aerosil® 380 F, Aerosil® 200 Pharma Aerosil® 300 Pharma, Aeropearl® 300/30, or Aeropearl® 300 Pharma.

In another embodiment, the composition comprises one or more pharmaceutically acceptable excipients. Pharmaceutical excipients useful for the compositions as described herein comprise: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); alkalizing agents (ammonia solution, ammonium carbonate, diethanolamine, diisopropanolamine, potassium hydroxide, sodium bicarbonate, sodium borate, sodium carbonate, sodium hydroxide, trolamine); antifoaming agents (dimethicone, simethicone); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, ascorbic acid, thimerosal, thymol); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); chelating agents (edetate disodium, ethylenediaminetetraacetic acid and salts, edetic acid); coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein); colorants (caramel, red, yellow, black or blends, ferric oxide); complexing agents (ethylenediaminetetraacetic acid and salts (EDTA), edetic acid, gentisic acid ethanolamide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); humectants (glycerol, hexylene glycol, sorbitol); plasticizers (e.g., castor oil, diacetylated monoglycerides, diethyl phthalate, glycerol, mono- and di-acetylated monoglycerides, propylene glycol, triacetin, triethyl citrate); polymers (e.g., cellulose acetate, alkyl celluloses, hydroxyalkyl, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); surfactants (simethicone); tablet binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); tablet disintegrants (alginic acid, microcrystalline cellulose, croscarmellose sodium, crospovidone, polacrilin potassium, sodium starch glycolate, starch, pregelatinized starch); tablet and/or capsule lubricants (calcium stearate, glyceryl behenate, magnesium stearate, light mineral oil, sodium stearyl fumarate, stearic acid, purified stearic acid, talc, hydrogenated vegetable oil, zinc stearate); thickening agents (gelatin having a bloom strength of 50-200); tonicity agent (dextrose, glycerol, mannitol, potassium chloride, sodium chloride); vehicle: flavoring or sweetening (aromatic elixir, compound benzaldehyde elixir, iso-alcoholic elixir, peppermint water, sorbitol solution, syrup, tolu balsam syrup); vehicle: oleaginous (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane); vehicle: solid carrier (sugar spheres); vehicle: sterile (bacteriostatic water for injection, bacteriostatic sodium chloride injection); viscosity-increasing (see suspending agent); water repelling agents (cyclomethicone, dimethicone, simethicone); or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol). This list is not meant to be exclusive, but instead merely representative of the classes of excipients and the particular excipients that may be used in oral dosage forms as described herein. See Remington's Essentials of Pharmaceutics, Pharmaceutical Press Publishing Company, London, UK, 1^{st} Edition, 2013, and the Handbook of Pharmaceutical Excipients, 8^{th} Edition, Pharmaceutical Press Publishing Company London, U K, 2017, which are incorporated by reference herein for such teachings.

In another embodiment, the composition includes a hydrophilic internal phase and a lipid or lipophilic external phase. The hydrophilic internal phase can comprise polypropylene glycol or polyethylene glycol of molecular weight ranging from about 200 to about 8000 (M_{N}, number average molecular weight). In another embodiment, the internal phase comprises hydroalcoholic solutions of cellulose derivatives, gelatins, or gelatin hydrolysates, polyacrylates, polyvinyl polymers, or combinations thereof. In one embodiment, the internal phase comprises polymers such as methylcellulose, hydroxypropylmethylcellulose, polymethylmethacrylate, or polyvinylpyrrolidone (PVP). In one embodiment, the internal phase of the composition state is “fluid” or “structured.” A “fluid” internal phase, as used herein, means a completely flowable liquid whose globules can aggregate to make a larger globule. A “structured” internal phase, as used herein, means a solid, semisolid, or a gel whose shape is relatively stable and does not usually aggregate to form a large globule. A structured internal phase can provide controlled drug release and stabilize the physical state of the composition. Without being bound to any theory, the structured nature of the composition impedes solvation or diffusion of the cannabinoid out of the composition.

In another embodiment, the composition is an emulsion type, where the cannabinoid is distributed in one or both of the external (lipophilic) and internal (hydrophilic) phases. The external phase of the emulsion composition comprises lipid or lipophilic vehicles similar to those described herein. The cannabinoid can be dispersed in the internal phase as a solution or as a suspension. For example, one portion of the cannabinoid in the form of a solid is incorporated in the internal phase, while another portion is dispersed in the external phase as an oil.

In one embodiment, the pharmaceutical composition described herein comprises a soft capsule comprising a composition comprising a lipid or lipophilic vehicle that provides a solid or semi-solid suspension of a cannabinoid.

In one embodiment, the cannabinoid comprises about 1% to about 50% of the composition by mass, including all integers and fractions within the specified range. In another embodiment, the cannabinoid comprises about 50%; about 45%; about 40%; about 35%; about 30%; about 25%, about 20%; about 15%; about 10%; about 5%; about 2%; or about 1% of the composition by mass. In one aspect, the cannabinoid comprises about 1-50% of the composition by mass. In another aspect, the cannabinoid comprises about 1-30% of the composition by mass. In another aspect, the cannabinoid comprises about 1-20% of the composition by mass. In another aspect, the cannabinoid comprises about 1-15% of the composition by mass. In another aspect, the cannabinoid comprises about 1-10% of the composition by mass. In another aspect, the cannabinoid comprises about 30% of the composition by mass. In another aspect, the cannabinoid comprises about 25% of the composition by mass. In another aspect, the cannabinoid comprises about 20% of the composition by mass.

In one embodiment, the cannabinoid-to-fill ratio range (e.g., the ratio of the cannabinoid mass to the mass of the other components of the matrix fill or vehicle) comprises from about 1:50 to about 1:1 by mass, including all ratios within the specified range. In another embodiment, the cannabinoid-to-fill ratio range comprises from about 1:10 to about 1:1 by mass, including all ratios within the specified range. In one aspect, the cannabinoid-to-fill ratio comprises about 1:9 to about 1:1 by mass, including all ratios within the specified range. In another aspect, the cannabinoid-to-fill ratio range comprises from about 1:5 to about 1:1 by mass, including all ratios within the specified range. In another aspect, the cannabinoid-to-fill ratio range comprises from about 1:3 to about 1:1.4 by mass, including all ratios within the specified range. In another aspect, the cannabinoid-to-fill ratio is about 1:5; about 1:4; about 1:3; about 1:2; about 1:1; or about 0.5:1. In other aspects, the cannabinoid-to-fill ratio is 1:3.5; 1:3.1; 1:2.9; 1:2.3; 1:2.5; 1:1.92; 1:1.77; 1:1.5; 1:1.4; 1:1.35; 1:1.2, or about 1:1.2.

In one embodiment, the low melting hard lipid comprises about 0% to about 50% of the composition by mass, including all integers and fractions within the specified range. In one embodiment, the low melting hard lipid comprises about 50%; about 45%; about 40%; about 35%; about 30%; about 25%, about 20%; about 15%; about 10%; about 5%; about 2%; or about 1% of the composition by mass. In one aspect, the low melting hard lipid comprises about 1-50% of the composition by mass. In another aspect, the low melting hard lipid comprises about 1-40% of the composition by mass. In another aspect, the low melting hard lipid comprises about 1-30% of the composition by mass. In another aspect, the low melting hard lipid comprises about 1-20% of the composition by mass. In another aspect, the low melting hard lipid comprises about 1-15% of the composition by mass. In another aspect, the low melting hard lipid comprises about 1-10% of the composition by mass. In another aspect, the low melting hard lipid comprises about 50% of the composition by mass. In another aspect, the low melting hard lipid comprises about 40% of the composition by mass. In another aspect, the low melting hard lipid comprises about 30% of the composition by mass. In another aspect, the low melting hard lipid comprises about 25% of the composition by mass. In another aspect, the low melting hard lipid comprises about 20% of the composition by mass. In another aspect, the low melting hard lipid comprises about 15% of the composition by mass.

As disclosed herein, the gelling agent may comprise one or more gelatins or gelatin hydrolysates. In one embodiment, the total amount of the gelling agent comprises about 1% to about 80% of the composition by mass, including all integers and fractions within the specified range. In another embodiment, the total amount of gelling agent comprises about 80%; about 75%; about 70%; about 65%; about 60%; about 55%; about 50%; about 45%; about 40%; about 35%; about 30%; about 25%, about 20%; about 15%; about 10%; about 5%; about 2%; or about 1% of the composition by mass. In one aspect, one or more gelling agents comprise about 60-80% of the composition by mass. In one aspect, one or more gelling agents comprise about 50-70% of the composition by mass. In one aspect, one or more gelling agents comprise about 40-60% of the composition by mass. In one aspect, one or more gelling agents comprise about 30-50% of the composition by mass. In another aspect, one or more gelling agents comprise about 20-40% of the composition by mass. In another aspect, one or more gelling agents comprise about 10-30% of the composition by mass. In another aspect, one or more gelling agents comprise about 5-20% of the composition by mass. In another aspect, one or more gelling agents comprise about 1-15% of the composition by mass. In another aspect, one or more gelling agents comprise about 1-10% of the composition by mass. In one aspect, gelatin of 50-100 Bloom comprises about 1-80% of the composition by mass including all integers and fractions within the specified range. In one aspect, gelatin hydrolysate comprises about 1-50% of the composition by mass including all integers and fractions within the specified range.

In one embodiment, the solvent comprises about 1% to about 40% of the composition by mass, including all integers and fractions within the specified range. In another embodiment, the gelling agent comprises about 40%; about 35%; about 30%; about 25%; about 20%; about 15%; about 10%; about 5%; about 2.5%; about 1%; or about 0.5% of the composition by mass. In one aspect, the solvent comprises quantum sufficiat (q.s.) to provide the total composition 100% by mass. In one aspect, the solvent is sesame oil, various vegetable oils, or a combination thereof.

In one embodiment, the antioxidant comprises about 0.001% to about 1% of the composition by mass, including all integers and fractions within the specified range. In another embodiment, the antioxidant comprises about 0.001%; about 0.005%; about 0.01%; about 0.05%; about 0.1%; about 0.2%, about 0.5%; or about 1% of the composition by mass. In one aspect, the antioxidant comprises about 0.05% of the composition by mass. In one aspect, the antioxidant comprises about 0.1% of the composition by mass. In one aspect, the antioxidant comprises about 0.2% of the composition by mass. In another aspect, the antioxidant comprises about 0.5% of the composition by mass.

In one embodiment, the composition comprises one of those shown in Table 2 including all possible iterations of the specified ranges that provide 100% total mass percentage.

TABLE 2
Exemplary Compositions

| | Mass percent (%) | | | |
|---|---|---|---|---|
| Ingredient | EX 1 | EX 2 | EX 3 | EX 4 |
| Cannabinoid (e.g., dronabinol in | 1-5 | 1-5 | 1-5 | 1-5 |
| sesame oil, ethanol, or Miglyol ® | | | | |
| 840) | | | | |
| Solvent (e.g., sesame oil) | 60-85 | 50-68 | 50-68 | 50-82 |
| Low-melting hard lipid (e.g., | 7.5-15 | 7.5-15 | 7.5-15 | 7.5-15 |
| Gelucire ® 43/01 and/or | | | | |
| Gelucire ® 53/13) | | | | |
| Antioxidant (e.g., BHA, BHT, | 0.1-15 | 0.1-15 | 0.1-15 | 0.1-15 |
| tocopherol, D/L-α-tocopherol, | | | | |
| Vitamin E, Vitamin E-TPGS, etc.) | | | | |
| Emulsifier | — | — | 1-5 | — |
| Optional other excipients | 0-10 | 0-10 | 0-10 | 0-10 |
| TOTAL | 100% | 100% | 100% | 100% |

In one embodiment described herein, the compositions described herein are encapsulated in a soft gelatin capsule under a nitrogen or helium atmosphere. In one embodiment, the soft capsule shell has the composition shown in Table 3, including all possible iterations of the specified ranges that provide 100% for the total mass percentage, including or excluding optional colorings, opacifiers, flavorings, or other excipients.

TABLE 3
Exemplary Soft Gelatin Capsule Composition

| Component | Exemplary Component | Mass percent (%) |
|---|---|---|
| Film-forming polymer | Gelatin, 150-200 Bloom | 20-48 |
| Plasticizer | Sorbitols | 10-30 |
| Solvent | Water | 20-70 |
| Opacifier (optional) | Titanium dioxide | 0-1.5 |
| Coloring agent (optional) | Various | 0-0.1 |
| Excipients (optional) | Various | 0-5 |
| TOTAL | | 100% |

Film-former polymers that are useful for creating soft capsules are gelatin, hydroxypropylmethylcellulose (HPMC) or carrageenan (e.g., iota carrageenan and kappa carrageenan). In one embodiment described herein, the film-forming polymer is gelatin.

Examples of gelatin compositions that are useful for creating soft capsule shells as described herein comprise acid bone gelatin, pig skin gelatin, chicken skin gelatin, fish gelatin, acid hide gelatin, gelatin hydrolysate, lime bone gelatin, or combinations thereof. Gelatins that are useful for creating soft capsules described herein can be classified as either Type A or Type B gelatin. Type A gelatin is derived from the acid hydrolysis of collagen (e.g., acid bone gelatin or pig skin gelatin), while Type B gelatin (e.g., lime bone gelatin) is derived from the alkaline hydrolysis of collagen. Traditionally, bovine bones and skins are used as raw materials for manufacturing Type A and Type B gelatin, while porcine skins are used extensively for manufacturing Type A gelatin. In addition, at neutral pH values, Type A gelatins (acid processed gelatins) are typically net cationic (e.g., isoelectric point of about 7-9) and Type B gelatins (alkali processed gelatins) are typically net anionic (e.g., isoelectric point of about 4.5-5.3). Type A gelatin typically has higher plasticity and elasticity than type B gelatin; type B gelatin typically has higher gel strength than type A gelatin.

The strength of gelatin compositions is typically defined by their Bloom strength or grade. The Bloom test determines the mass (in grams) needed by a 0.5-inch diameter probe to deflect the surface of a gel 4 mm without breaking it. The result is expressed as “Bloom” or “Bloom strength.” The soft capsules described herein utilize gelatins with Bloom strengths in the range of about 20 Bloom to about 400 Bloom, including each integer within the specified range. In one embodiment, Bloom strengths for soft capsules described herein are about 50 Bloom to about 250 Bloom including each integer within the specified range. In some embodiments, the gelatin Bloom strength is about 50 Bloom, about 80 Bloom, about 100 Bloom, about 120 Bloom, about 150 Bloom, about 180 Bloom, about 200 Bloom, or about 250 Bloom. In one embodiment, the gelatin Bloom strength is 100 Bloom. In another embodiment, the gelatin Bloom strength is 150 Bloom. In another embodiment, the gelatin Bloom strength is 195 Bloom. In another embodiment, the gelatin Bloom strength is 200 Bloom.

Plasticizers that are useful for creating soft capsules as described herein are sorbitol, partially dehydrated sorbitol (a blend of D-sorbitol, 1,4-sorbitan, mannitol, and water; e.g., Sorbitol Special® (SPI Pharma); Anidrisorb® or Polysorb®, (Roquette)), maltitol (hydrogenated corn syrup; e.g., Lycasin®, Roquette), corn syrup, xylitol, mannitol, propylene glycol, low molecular weight polyethylene glycols, poly-alcohols with 3 to 6 carbon atoms, or a combination thereof. Plasticizers typically comprise about 10-30% of the total wet mass of a shell, including each integer within the specified range. The mass ratio between the film-forming polymer, plasticizer, and solvent is adjusted so that the gel mass is flowable and not too viscous and can be made into soft capsules using rotary die encapsulation methods. In one embodiment, the plasticizer is a sorbitol.

In one embodiment described herein, the soft capsule shell has the exemplary composition shown in Table 4.

TABLE 4
Exemplary Soft Capsule Shell Composition

| Component | Mass Percent (%) |
|---|---|
| Film forming polymer (e.g., gelatin) | 20-50 |
| Plasticizer (e.g., sorbitol, g., | 15-30 |
| Polysorb ® 85/70/00; Roquette) | |
| Solvent (e.g., water) | q.s. (e.g., 20-40%) |
| TOTAL | 100% |
| Final pH | ~4-7 |
| Ratio total plasticizer to gelatin | 20:43 (0.46:1) |
| Water content in dried soft capsule shell: | 8-15% |

In one embodiment, the mass percentage range of film-forming polymer of the soft capsule described herein is about 20% to about 50%, including all integers within the specified range. In one aspect, the film-forming polymer mass percentage is about 38%. In another aspect, the film-forming polymer mass percentage is about 42%. In another aspect, the film-forming polymer mass percentage is about 44%.

In one embodiment, the mass percentage range of plasticizer is about 15% to about 30%, including all iterations of integers with the specified range. In one aspect, the plasticizer mass percentage is about 24%. In another aspect, the plasticizer mass percentage is about 22%. In another aspect, the plasticizer mass percentage is about 20%.

In one embodiment, the solvent comprises about 20% to about 40% of the soft capsule composition, including all integers and fractions within the specified range. In one embodiment, the solvent is water. The quantity of water in the composition varies depending on the quantities of the other ingredients. For example, the quantity of plasticizer, opacifier, colorant, flavoring, or other excipients can change the percentage of water present in the composition. In one embodiment, the mass percentage of water is as much as suffices to bring the total mass percentage to 100% (i.e., quantum sufficiat; q.s.). In another embodiment, the water comprises about 20%, about 25%, about 30%, about 35%, or about 40% of the enteric soft capsule composition. In another embodiment, water comprises about 30% to about 40% of the enteric soft capsule composition. In one embodiment, water comprises about 34% of the composition.

In one embodiment, the final moisture (water) content of the soft capsule after manufacturing and drying is from about 8% to about 15%, including all integers and fractions within the specified range. In another embodiment, the moisture content is about 8% to about 12%, including all integers and fractions within the specified range. In one aspect, the final moisture content is about 12%. In one aspect, the final moisture content is about 11%. In one aspect, the final moisture content is about 10%. In one aspect, the final moisture content is about 9%. In another aspect, the final moisture content is about 8%.

In one embodiment, the mass percentage ratio range of plasticizer to film-forming polymer is about 0.33:1 to about 0.56:1, including all iterations of iterations of ratios with the specified range. In one embodiment, the mass percentage ratio range of plasticizer to film-forming polymer is about 0.38:1. In one embodiment, the mass percentage ratio range of plasticizer to film-forming polymer is about 0.42:1. In one embodiment, the mass percentage ratio range of plasticizer to film-forming polymer is about 0.46:1. In one embodiment, the mass percentage ratio range of plasticizer to film-forming polymer is about 0.52:1.

In one embodiment described herein, the soft capsule comprises about 42% of at least one film-forming polymer; about 24% of at least one plasticizer; and about 34% water.

In another embodiment, the soft capsule shell has the exemplary composition shown in Table 5.

TABLE 5
Exemplary Soft Capsule Shell Composition

| Component | Mass Percent (%) |
|---|---|
| Gelatin, 150 Bloom, Lime Bone or Acid Bone | 42 |
| Sorbitol (e.g., Polysorb ® 85/70/00; Roquette) | 24 |
| Water | 34 |
| TOTAL | 100% |

In one aspect, soft capsules are made using a rotary die apparatus as described in U.S. Pat. Nos. 5,459,983; 5,146,730; and 6,482,516, each of which are incorporated by reference herein for such teachings.

Another embodiment described herein includes a process of manufacturing soft capsules comprising any of the pharmaceutical composition as described herein. The process includes preparing a gel mass composition comprising a film-forming, water-soluble polymer, an appropriate plasticizer, and solvent; casting the gel mass into films or ribbons using heat-controlled drums or surfaces; and manufacturing a soft capsule comprising a matrix fill using rotary die technology. The thickness of the films or ribbons that form the soft capsule shell is from about 0.010 inches (≈0.254 mm) to about 0.050 inches (≈1.27 mm), including all integers within the specified range. The shell thickness can be about 0.010 inch (≈0.254 mm), about 0.015 inch (≈0.381 mm), about 0.02 in (≈0.508 mm), about 0.03 in (≈0.762 mm), about 0.04 in (≈1.02 mm), or about 0.05 in (≈1.27 mm). In one embodiment, the thickness is about 0.02 inches (≈0.508 mm) to about 0.040 inches (≈1.02 mm). In one embodiment, the shell thickness is about 0.028 inches (≈0.711 mm). In another embodiment, the shell thickness is about 0.033 inches (≈0.838 mm). In another embodiment, the shell thickness is about 0.038 inches (≈0.965 mm). In another embodiment, the shell thickness is about 0.035 inches (≈0.889 mm). In another embodiment, the shell thickness is about 0.038 inches (≈0.965 mm). In another embodiment, the shell thickness is about 0.040 inches (≈1.02 mm).

In one embodiment described herein, the soft capsule shell described herein, encapsulates a matrix fill as described herein. In another embodiment described herein, the soft capsule shell and encapsulated matrix fill comprises an outer dimension from about 2 oval to about 30 oval including all iterations of capsule size within the specified range (e.g., 2 oval, 3 oval, 4 oval, 5 oval, 6 oval, 7 oval, 8 oval, 10 oval, 12 oval, 16 oval, 20, or 30 oval). In another embodiment described herein, the soft capsule shell and encapsulated matrix fill comprises an outer dimension from about 2 round to about 28 round including all iterations of capsule size within the specified range (e.g., 2 round, 3 round, 4 round, 5 round, 6 round, 7 round, 8 round, 10 round, 12 round, 16 round, 20 round or 28 round). In another embodiment described herein, the soft capsule shell and encapsulated matrix fill comprises an outer dimension from about 2 oblong to about 22 oblong including all iterations of capsule size within the specified range (e.g., 2 oblong, 3 oblong, 4 oblong, 5 oblong, 6 oblong, 7 oblong, 8 oblong, 10 oblong, 11, oblong, 12 oblong, 14 oblong, 16 oblong, 20 oblong, or 22 oblong). Dimension specifications of soft capsules and tablets are known to those skilled in the art. See Remington's Essentials of Pharmaceutics, Pharmaceutical Press Publishing Company, London, UK, 1^{st} Edition, 2013, which is incorporated by reference herein for such teachings.

In one embodiment described herein, soft capsules are coated with a coating comprising the exemplary composition shown in Table 6.

TABLE 6
Exemplary Coating Composition

| | | Mass |
|---|---|---|
| Component | Exemplary Component | Percent (%) |
| Optional Enteric | Methacrylic acid copolymers, | 5-90 |
| Polymer(s) | polyvinyl acetate phthalates, | |
| | polyvinyl phthalate, cellulose | |
| | acetate phthalates, cellulose | |
| | acetate trimellitate, cellulose | |
| | acetate succinate, | |
| | hydroxypropyl | |
| | methylcellulose, | |
| | carboxymethyl cellulose | |
| Plasticizer(s) | Triethyl citrate, tributyl citrate, | 0-25 |
| | polyethylene glycols, | |
| | propylene glycol, triacetin, | |
| | dibutyl phthalate, tripropionin, | |
| | ethyl acid phtalate, butyl acid | |
| | phthalate, ethyl acid adipate, | |
| | fats and waxes mixed with | |
| | esters, glycerin | |
| Neutralizing | Ammonia, NaOH, sodium | 0-5 |
| agent | bicarbonate | |
| Solubilizers | Sodium lauryl sulfate, sodium | |
| | lauroyl sarcosinate sodium | |
| | dodecyl sulfate, polysorbate | |
| | 20, polysorbate 80, other | |
| | detergents and surfactants | |
| Solvent(s) | Water, ethanol, isopropanol, | 50-80 |
| | acetone | |
| Excipients | Emulsifiers, pore-forming | 0-20 |
| | agents, anti-adherents, | |
| | surfactants, pigments, | |
| | colorants, antifoam, | |
| | antioxidants, waxes, | |
| | magnesium stearate, | |
| | micronized amorphous silica, | |
| | kaolin, talc, | |

Enteric polymers useful for enteric coatings include pH-dependent polymers that are less soluble in an aqueous media with acidic pH and more soluble in an aqueous media with basic pH. In one embodiment, the enteric of pH dependent material dissolves or rapidly disperses at a pH level above pH 5.0, above pH 5.5, or above pH 6.0.

Exemplary enteric polymers useful for coats include cellulose acetate phthalate, hydroxypropylmethyl cellulose phthalate, hydroxypropyl methylcellulose acetate succinate, polyvinyl acetate phthalate, cellulose acetate trimellitate, carboxymethylcellulose, methacrylic acid copolymers such as, Eudragit L (polymethacrylic acid, methylmethacrylate, 1:1 ratio), or Eudragit S (polymethacrylic acid, methylmethacrylate, 1:2 ratio), shellac, zein, or combinations thereof.

Suitable plasticizers include acetyl triethyl citrate, dibutyl phthalate, tributyl citrate, triethyl citrate, acetyl tributyl citrate, propylene glycol, triacetin, polyethylene glycol, diethyl phthalate, or combinations thereof.

Suitable solubilizers include sodium lauryl sulfate, sodium lauroyl sarcosinate sodium dodecyl sulfate, polysorbate 20, polysorbate 80, other detergents or surfactants, or combinations thereof.

Anti-adherent agents serve to prevent potential agglomeration in acid media. Suitable anti-adherents include talc, magnesium stearate, calcium stearate, stearic acid, hydrogenated vegetable oils, polyethylene glycols, fumed silica, silicon dioxide, or combinations thereof.

Pore-forming agents serve to create pores or channels in the enteric coating after administration to a human. Suitable pore-forming agents include sodium chloride, potassium chloride, sucrose, sorbitol, mannitol, polyethylene glycols (PEG), propylene glycol, hydroxypropyl cellulose, hydroxypropyl methylcellulose, polyvinyl alcohols, methacrylic acid copolymers, poloxamers, or combinations thereof.

Many conventional coating excipients are described in the art. See e.g., Rowe et al., Eds. Handbook of Pharmaceutical Excipients, 7^{th} ed. Royal Pharmaceutical Society, UK (2012).

In one embodiment described herein, the enteric coating comprises methacrylic acid and ethyl acrylate copolymer (e.g., EUDRAGIT® L100-55, Evonik), talc, triethyl citrate, sodium bicarbonate, colloidal silica, sodium lauryl sulfate, and water.

In one embodiment, adjusting the amount of enteric coating and the ratio of polymer to other components allows for tuning the release profile of the dosage form.

Subcoats can be applied to the soft capsules prior to coating to prevent shell-coat interactions and improve coating adhesion to the capsule. Exemplary subcoatings can comprise polyvinylpyrrolidone, polyvinyl alcohols, hydroxypropyl methylcellulose, polyethylene glycol, oils, or combinations thereof.

Coatings, top coatings, or subcoatings are applied to the soft capsules using various methods know in the art. The coatings are typically prepared as suspensions and sprayed on capsules in perforated coating pans through one or more spray nozzles at a specific temperature. Coating solutions or dispersion may be applied at spray rates between 100 and 400 g/min. The spray rate may be proportionately higher for coatings with higher solid content and lower for more dilute dispersions. In one embodiment, capsules are coated using a pan coater. After the enteric coating suspension is applied, the coated capsules are dried in the pan coater for a period of time at a specific temperature.

Another embodiment described herein comprises a subcoating that is applied prior to applying an enteric coating. In one embodiment, the subcoating comprises hydroxypropyl methylcellulose, methylcellulose, ethylcellulose, or a combination thereof.

Another embodiment described herein comprises a moisture barrier that is applied as a top coating on the enteric coating. In one embodiment the moisture barrier comprises one or more polyvinyl alcohols and appropriate pharmaceutically acceptable excipients. In one embodiment the moisture barrier comprises polyvinyl alcohol, sodium lauryl sulfate, glyceryl mono-caprylate-caprate, and talc. In one aspect, the moisture barrier aids in preventing the ingress of oxygen or moisture. In another aspect, the moisture barrier aids in preserving the cosmetic appearance of the dosage forms by preventing dimpling, sticking, or other processing or storage induced blemishes.

The pharmaceutical compositions described herein can comprise soft gelatin capsules encapsulating a matrix fill that is semi-solid or solid. The fill is molten during manufacturing but solidifies after cooling and drying owing to both the gelling agent and the low melting hard lipid. Without being bound by any theory, it is believed that the solid or semisolid cannabinoid suspension described herein limits oxygen and water vapor ingress into the capsule fill and prevents oxidation or degradation of the cannabinoid API. Further, the low melting hard lipid protects the API from oxidation and humidity. Antioxidants in the fill additionally protect the API from oxidation. The capsules can be coated to further limit exposure to air, moisture, and light.

### Methods of Manufacturing

Another embodiment described herein is a method for manufacturing the pharmaceutical composition described herein comprising the steps of: (a) preparing a fill composition under nitrogen gas or helium comprising combining a cannabinoid (e.g., dronabinol); a gelling agent, an optional low-melting hard lipid, an optional antioxidant, and a solvent and mixing to homogenize; heating the fill composition at 30-70° C. until all components are dissolved; (b) preparing a gel mass composition comprising one or more film forming polymers, one or more plasticizers, one or more excipients and one or more solvents; (c) casting the gel composition into films or ribbons using heat-controlled drums or surfaces; and (d) forming a soft gel dosage form comprising a matrix fill using rotary die encapsulation technology. Another embodiment is a pharmaceutical dosage form manufactured by the manufacturing method.

### Dosages

In one embodiment, the pharmaceutical compositions described herein contain doses of either 2.5 mg, 5 mg, or 10 mg of a cannabinoid such as dronabinol. In one embodiment, the dosage depends on the indication. In one embodiment, for the stimulation of appetite, the dosage is dosage of dronabinol is 2.5 mg to 20 mg/day. Typically, 2.5 mg of dronabinol is administered as two doses per day (BID), three doses per day (TID), or four doses per day (QID) to achieve a total daily dosage of 5-10 mg/day. In severe cases, 5 mg per dose can be administered. In another embodiment, any of the foregoing doses comprise a total daily dosage.

In another embodiment, for use as an antiemetic in the treatment of nausea or vomiting, the dosage is 5 mg to 20 mg/day. Typically, 5 mg of dronabinol is administered as two doses per day (BID), three doses per day (TID), or four doses per day (QID) to achieve a total daily dosage of 10-20 mg/day. In another embodiment, any of the foregoing doses comprise a total daily dosage.

In one embodiment, the pharmaceutical compositions described herein provide a dosage form of the pharmaceutical compositions described here for administration to a subject. In one embodiment, the subject is suffering from or has the symptoms of anorexia, nausea, vomiting, or other gastrointestinal symptoms. The dosage form can be administered, for example, to a subject, or a subject in need thereof. In one aspect, the subject is a mammal, or a mammal in need thereof. In one aspect, the subject is a human, or human in need thereof. In one aspect, the subject is a human or a human in need thereof. In one aspect, the subject is a child (˜0-9 years old) or an adolescent (˜10-17 years old). In one aspect, the subject is from about 0 to about 9 years of age. In another aspect, the subject is from about 10 years to about 17 years of age. In another aspect, the subject is over 17 years of age. In another aspect, the subject is an adult (18 years of age).

In one embodiment, the subject is a human infected with Human Immunodeficiency Virus (HIV). In another embodiment, the subject is a human having Acquired Immunodeficiency Syndrome (AIDS). In another embodiment, the subject has either HIV or AIDS and has anorexia or weight loss due to lack of appetite. In one aspect, the subject is an AIDS patient having anorexia associated with weight loss.

In another embodiment, subject is a human having cancer or other disease or disorder that is being treated with chemotherapy or radiation treatments that is associated with nausea or vomiting. In one aspect, the subject is a cancer chemotherapy patient who has failed to respond adequately to conventional antiemetic treatments.

One or more dosage forms of the compositions described herein can be administered, for example, 1×, 2×, 3×, 4×, 5×, 6×, or even more times per day. One or more dosage forms can be administered, for example, for 1, 2, 3, 4, 5, 6, 7 days, or even longer. One or more dosage forms can be administered, for example, for 1, 2, 3, 4 weeks, or even longer. One or more dosage forms can be administered, for example, for 1, 2, 3, 4, 5, 6, 7, 8, 9, 10, 11, 12 months, 1 year, 2, years, 3 years, 4 years, 5 years, over 5 years, a decade, multiple decades, or even longer. One or more dosage forms can be administered at a regular interval until the subject or subject in need thereof, does not require treatment, prophylaxis, or amelioration of any disease or condition including but not limited to anorexia associated with weight loss in patients with AIDS and nausea and vomiting associated with cancer chemotherapy in patients who have failed to respond adequately to conventional antiemetic treatments.

In one embodiment, the compositions described herein can be administered as dosage forms in various regimens, including one dose per day (QD), two doses per day (BID), three doses per day (TID), or four times per day (QID) to achieve a total daily dosage. In another embodiment, any of the foregoing doses comprise a total daily dosage.

Another embodiment described herein is a method of treating a subject suffering from or having the symptoms of anorexia associated with weight loss in patients with AIDS and nausea and vomiting associated with cancer chemotherapy by orally administering one or more of the pharmaceutical compositions described herein to the subject. The composition may be administered in one or more doses, one or more times per day for a total daily dosage.

Another embodiment described herein is a method of treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders by orally administering one or more of the pharmaceutical compositions described herein to the subject. The composition may be administered in one or more doses, one or more times per day for a total daily dosage.

Toxicity and therapeutic efficacy of compounds described herein, including pharmaceutically acceptable salts and deuterated variants, can be determined by standard pharmaceutical procedures in cell cultures or experimental animals. The LD₅₀ is the dose lethal to 50% of the population. The ED₅₀ is the dose therapeutically effective in 50% of the population. The dose ratio between toxic and therapeutic effects (LD₅₀/ED₅₀) is the therapeutic index. Compounds that exhibit large therapeutic indexes are preferred. While compounds that exhibit toxic side effects may be used, care should be taken to design a delivery system that targets such compounds to the site of affected tissue in order to minimize potential damage to uninfected cells and thereby reduce side effects.

Data obtained from cell culture assays and animal studies can be used in formulating a range of dosage for use in humans. The dosage of such compounds may lie within a range of circulating concentrations that include the ED₅₀ with little or no toxicity. The dosage may vary within this range depending upon the dosage form employed and the route of administration utilized. For any compound, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography of a blood or plasma sample.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

### Methods of Use

Another embodiment described herein is a method of use of the pharmaceutical compositions described herein. In one aspect, the use comprises treating a subject suffering from or having the symptoms of anorexia associated with weight loss in AIDS and nausea and vomiting associated with cancer chemotherapy by orally administering one or more of the pharmaceutical compositions described herein to the subject. The composition can comprise dronabinol as described herein. The composition may be administered in one or more doses, one or more times per day for a total daily dosage.

Another embodiment described herein is a composition for use in treating a subject suffering from or having the symptoms of anorexia associated with weight loss in AIDS or nausea and vomiting associated with cancer chemotherapy. The composition can comprise dronabinol as described herein. The composition may be administered in one or more doses, one or more times per day for a total daily dosage.

Another embodiment described herein is a method of use of the pharmaceutical compositions described herein. In one aspect, the use comprises treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders by orally administering one or more of the pharmaceutical compositions described herein to the subject. The composition can comprise dronabinol as described herein. The composition may be administered in one or more doses, one or more times per day for a total daily dosage.

Another embodiment described herein is a composition for use in treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders. The composition can comprise dronabinol as described herein. The composition may be administered in one or more doses, one or more times per day for a total daily dosage.

### Combination Therapies

Another embodiment is a pharmaceutical combination comprising the pharmaceutical dosage form comprising a cannabinoid described herein, and one or more additional therapeutic agent(s) for simultaneous, separate, or sequential use in therapy for anorexia associated with weight loss in AIDS patients or nausea and vomiting associated with cancer chemotherapy in patients who have failed to respond adequately to conventional antiemetic treatments. Potential combination therapeutics include antivirals, antibiotics, chemotherapeutics, radiation treatments, biologics, antacids, antiemetics, nutritional supplements, analgesics, NSAIDs, cimetidine, ranitidine, famotidine, ondansetron, omeprazole, lansoprazole, rabeprazole, esomeprazole, pantoprazole, inter alia.

### Kits

Another embodiment described herein is a kit comprising the pharmaceutical dosage form in an oxygen-impermeable a tamper evident packaging. The term “tamper evident” or “tamper resistant” refers to a packaging of any kind that readily displays or permits an individual to observe any physical interference or manipulation of said packaging. The tamper evident packaging provides reasonable evidence to consumers that tampering has occurred. The tamper evident packaging additionally contains appropriate labelling statements describing the features and evidences of the tamper evident packaging. In one aspect, the tamper evident packaging comprises: bottles, film wrappers, blister or strip packs, bubble packs, heat shrink bands or wrappers, foil, paper, or plastic pouches, container mouth inner seals, tape seals, breakable caps, sealed metal tubes or plastic heat-sealed tubes, sealed cartons, aerosol containers, cans including metal and composite materials, or any combination thereof. The packaging may also comprise a desiccant, an oxygen absorber, an inert atmosphere such as nitrogen or helium, and packing filler material to prevent the contents from shifting or rattling. The packaging may also contain appropriate instructions for prescribing, instructions for use, warnings, or other appropriate information. In another aspect, the packaging may contain at least one daily regimen for the oral pharmaceutical composition.

Another embodiment described herein is a kit for dispensing any of the oral pharmaceutical compositions described herein comprising: (a) at least one oral pharmaceutical dosage from; (b) at least one oxygen and moisture proof dispensing receptacle comprising blister or strip packs, an aluminum blister, a transparent or opaque polymer blister with pouch, polypropylene tubes, colored blister materials, tubes, bottles, and bottles optionally containing a child-resistant feature, optionally comprising a desiccant, such as a molecular sieve or silica gel, an inert atmosphere such as nitrogen or helium, an oxygen absorber; and optionally (c) at least one daily regimen for the oral pharmaceutical composition; and (d) an insert comprising instructions or prescribing information for the oral pharmaceutical composition. In one aspect described herein, the kit is useful for treating anorexia or nausea or vomiting according to any of the methods described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

Exemplary compositions as described herein are shown in Table 7. Composition components are set forth by mass percentage of the total weight of the composition. Such compositions cab be encapsulated in soft gelatin capsules using rotary die encapsulation as described herein. Exemplary soft gelatin capsule formulations are shown in Table 8. Exemplary compositions as described herein are shown in Table 9.

TABLE 7
Exemplary Compositions

| | Mass Percent (%) | | | |
|---|---|---|---|---|
| Ingredient | EX 1 | EX 2 | EX 3 | EX 4 |
| Cannabinoid (e.g., dronabinol in | 1-20 | 1-20 | 1-30 | 1-20 |
| sesame oil, Miglyol ® 840, or | | | | |
| ethanol) | | | | |
| Gelling agent (e.g., hydrolyzed | 1-10 | 1-10 | 1-50 | 1-10 |
| gelatin) | | | | |
| Gelling agent (e.g., gelatin 50-100 | 15 | 50 | 1-80 | 1-75 |
| Bloom) | | | | |
| Low-melting hard lipid (e.g., | 35 | — | 1-50 | — |
| Gelucire ® 43/01) | | | | |
| Antioxidant (e.g., BHA or BHT, et | 0.001 | — | 0-0.05 | 0-0.05 |
| al.) | | | | |
| Solvent (e.g., water or sorbitol) | 20 | 20 | 5-30 | 2 |
| Optional other excipients | 0-10 | 0-10 | 0-10 | 0-10 |
| TOTAL | 100% | 100% | 100% | 100% |

TABLE 8
Exemplary Soft Capsule Shell Composition

| Component | Mass Percent (%) |
|---|---|
| Film forming polymer (e.g., gelatin) | 20-50 |
| Plasticizer (e.g., sorbitol, g., | 15-30 |
| Polysorb ® 85/70/00; Roquette) | |
| Solvent (e.g., water) | q.s. (e.g., 20-40%) |
| TOTAL | 100% |
| Final pH | ~4-7 |
| Ratio total plasticizer to gelatin | 20:43 (0.46:1) |
| Water content in dried soft capsule shell: | 8-15% |

TABLE 9
Exemplary Compositions

| | Mass | Mass | Mass | Mass | Mass | Mass |
|---|---|---|---|---|---|---|
| Component | (mg) | % | (mg) | % | (mg) | % |
| Cannabinoid (e.g., dronabinol | 2.5 | 1.7% | 5 | 3.3% | 10 | 6.7% |
| in sesame oil, Miglyol ® 840, or | | | | | | |
| ethanol) | | | | | | |
| Gelling agent (e.g., hydrolyzed | 70 | 46.5% | 70 | 46.6% | 70 | 46.6% |
| gelatin/gelatin) | | | | | | |
| Low-melting hard lipid (e.g., | 45 | 29.9% | 45 | 30.0% | 45 | 30.0% |
| Gelucire ® 43/01) | | | | | | |
| Antioxidant (e.g., BHA or BHT, | 0.1 | 0.1% | 0.1 | 0.1% | 0.1 | 0.1% |
| et al.) | | | | | | |
| Solvent (e.g., water or sorbitol) | 33 | 21.9% | 30 | 20.0% | 25 | 16.7% |
| Optional other excipients | — | — | — | — | — | — |
| Total | 150.6 | 100% | 150.1 | 100% | 150.1 | 100% |
| Cannabinoid (e.g., dronabinol | 2.5 | 1.2% | 5 | 2.5% | 10 | 5.0% |
| in sesame oil, Miglyol ® 840, or | | | | | | |
| ethanol) | | | | | | |
| Gelling agent (e.g., hydrolyzed | 98 | 48.9% | 98 | 49.0% | 98 | 49.0% |
| gelatin/gelatin) | | | | | | |
| Low-melting hard lipid (e.g., | 60 | 29.9% | 60 | 30.0% | 60 | 30.0% |
| Gelucire ® 43/01) | | | | | | |
| Antioxidant (e.g., BHA or BHT, | 0.1 | 0.0% | 0.1 | 0.0% | 0.1 | 0.0% |
| et al.) | | | | | | |
| Solvent (e.g., water or sorbitol) | 40 | 19.9% | 37 | 18.5% | 32 | 16.0% |
| Optional other excipients | — | — | — | — | — | — |
| Total | 200.6 | 100% | 200.1 | 100% | 200.1 | 100% |

### Example 2

Dosage forms were manufactured in a closed system under a nitrogen or helium atmosphere to reduce oxygen exposure. The fill composition was prepared by combining the dronabinol API (solubilized in sesame oil or Miglyol® 840) with hydrolyzed gelatin and water or sorbitol. Add gelatin of 50-100 Bloom and homogenize. Add a low-melting hard lipid (e.g., Gelucire® 43/01) and homogenize with heating to 30-60° C. until all components are dissolved. Water or sorbitol can be added to decrease viscosity and improve flowability of the composition.

The capsule shell gel mass was prepared by combining gelatin, sorbitol and water and heating the composition to 50-70° C. until the composition is molten and flowable. The gel may be aged. The gel was then casted into films or ribbons using heat-controlled drums or surfaces. The soft gelatin capsules were formed using rotary die encapsulation technology where the fill is injected between two gelatin films while simultaneously being struck in a die to form soft capsules. The capsules were dried under controlled conditions. Upon drying the fill became a solid or semisolid gel.

The capsules were packaged in bottles of 60 under an inert atmosphere with optional oxygen and moisture scavenging package inserts. Alternatively, the capsules can be packaged in 10-count blister packages.

### Example 3

Dissolution testing was performed using USP and FDA guidelines. Dissolution was performed using United States Pharmacopeia (USP) Method [symbol] 711 [symbol] Dissolution with Apparatus 2 (Paddle Apparatus) at 100-150 rpm in water with 10% Labrasol®. USP Method [symbol] 711 [symbol] Dissolution (Dec. 1, 2011) is incorporated by reference herein for such teachings. Samples were removed at 0, 5, 10, 15, 30, 45, and 60-minute intervals until at least 80% of the content was released. Drug release is analysed using HPLC.

USP Capsule rupture testing was performed.

Dissolution and rupture testing were compared to MARINOL® capsules as a control.

### Example 5

Dosage forms were tested for stability at 50° C. for 1 or 2 weeks. Further tests included stability at 25° C.±2° C. and 60% relative humidity (RH)±5% RH for 1, 6, 12, 18, and 24 months and 40° C.±2° C. and 75% RH±5% RH for 1, 2, 3, and 6 months. Stability was compared to MARINOL® capsules as a control.

The results showed that the initial formulations containing a gelling agent were not acceptable. The gelatin was not miscible with sesame oil and over time they would be separate phase. That would cause an issue with content uniformity because the mix should be constantly mixed during encapsulation. The gelling agent was removed, and new formulations were developed.

### Example 6

The following formulations were prepared and screened for stability and evaluated using viscosity analysis and differential scanning calorimetry for melting behavior. The formulations contain 5 mg of dronabinol. The 10 mg strength contains double the 5 mg amount of dronabinol, and the 2.5 mg strength contains half of the 5 mg amount with the difference adjusted by the removal or addition of the sesame oil solvent, respectively.

TABLE 10
Summary of Dronabinol Formulations

| | Formulations (Mass %) | | | | | | | | | | | | | |
|---|---|---|---|---|---|---|---|---|---|---|---|---|---|---|
| Component | 1 | 2 | 3 | 4 | 5 | 6 | 7 | 8 | 9 | 10 | 11 | 12 | 13 | 14 |
| Dronabinol | 2.9 | 2.9 | 2.9 | 2.9 | 2.9 | 2.9 | 2.9 | 2.9 | 2.9 | 2.9 | 2.9 | 3.0 | 3.0 | 3.0 |
| Sesame oil | 81.9 | 81.9 | 82.0 | 82.0 | 77.1 | 77.1 | 81.9 | 82.0 | 77.1 | 72.1 | 72.1 | 67.0 | 67.0 | 82.0 |
| Hard fat | — | 15.0 | — | 15.0 | — | 15.0 | 7.5 | 7.5 | 7.5 | 15.0 | 15.0 | 15.0 | 15.0 | 15.0 |
| thickening | | | | | | | | | | | | | | |
| agent (e.g., | | | | | | | | | | | | | | |
| Gelucire ® | | | | | | | | | | | | | | |
| 43/01) | | | | | | | | | | | | | | |
| Hard fat | 15.0 | — | 15.0 | — | 15.0 | — | 7.5 | 7.5 | 7.5 | — | — | — | — | — |
| thickening | | | | | | | | | | | | | | |
| agent (e.g., | | | | | | | | | | | | | | |
| Gelucire ® | | | | | | | | | | | | | | |
| 50/13) | | | | | | | | | | | | | | |
| Propylene | — | — | 0.1 | 0.1 | — | — | — | 0.1 | — | — | — | — | — | — |
| gallate | | | | | | | | | | | | | | |
| D/L-α- | — | — | — | — | 5.0 | 5.0 | — | — | 5.0 | 5.0 | — | 15.0 | — | — |
| tocopherol | | | | | | | | | | | | | | |
| BHT | 0.2 | 0.2 | — | — | — | — | 0.2 | — | — | — | — | — | — | — |
| Vit E TPGS | — | — | — | — | — | — | — | — | — | — | 5.0 | — | 10.0 | — |
| Lecithin | — | — | — | — | — | — | — | — | — | 5.0 | 5.0 | — | 5.0 | — |
| TOTAL | 100 | 100 | 100 | 100 | 100 | 100 | 100 | 100 | 100 | 100 | 100 | 100 | 100 | 100 |

The mass and mass percentage of the formulations shown in Table 10 are listed in Table 11. In the formulations, “DRB/Sesame oil,” refers to the stock solution of dronabinol in sesame oil containing 0.2 mg/mL dronabinol.

TABLE 11
Dronabinol Formulations (5 mg of Dronabinol)

| | | |
|---|---|---|
| Component | Mass % | mg/sg |

Formulation 1

| | | |
|---|---|---|
| DRB/Sesame oil | 14.3% | 25 |
| Sesame oil | 70.5% | 123.4 |
| Gelucire ® 50/13 | 15.0% | 26.25 |
| BHT | 0.2% | 0.35 |
| Total | 100% | 175 |
| Strength (mg/g) | | 28.57 |
| Formulation 2 | | |
| DRB/Sesame oil | 14.3% | 25 |
| Sesame oil | 70.5% | 123.4 |
| Gelucire ® 43/01 | 15.0% | 26.25 |
| BHT | 0.2% | 0.35 |
| Total weight (mg) | | 175 |
| Strength (mg/g) | | 28.57 |

Formulation 3

| | | |
|---|---|---|
| DRB/Sesame oil | 14.3% | 25 |
| Sesame oil | 70.6% | 123.575 |
| Gelucire ® 50/13 | 15.0% | 26.25 |
| Propylene gallate | 0.1% | 0.175 |
| Total | 100% | 175 |
| Strength (mg/g) | | 28.57 |

Formulation 4

| | | |
|---|---|---|
| DRB/Sesame oil | 14.3% | 25 |
| Sesame oil | 70.6% | 123.575 |
| Gelucire ® 43/01 | 15.0% | 26.25 |
| Propylene gallate | 0.1% | 0.175 |
| Total | 100% | 175 |
| Strength (mg/g) | | 28.57 |

Formulation 5

| | | |
|---|---|---|
| DRB/Sesame oil | 14.3% | 25 |
| Sesame oil | 65.7% | 115 |
| D/L-α-tocopherol | 5.0% | 8.75 |
| Gelucire ® 50/13 | 15.0% | 26.25 |
| Total | 100% | 175 |
| Strength (mg/g) | | 28.57 |

Formulation 6

| | | |
|---|---|---|
| DRB/Sesame oil | 14.3% | 25 |
| Sesame oil | 65.7% | 115 |
| D/L-α-tocopherol | 5.0% | 8.75 |
| Gelucire ® 43/01 | 15.0% | 26.25 |
| Total | 100% | 175 |
| Strength (mg/g) | | 28.57 |

Formulation 7

| | | |
|---|---|---|
| Gelucire ® 50/13 | 7.5% | 13.125 |
| Gelucire ® 43/01 | 7.5% | 13.125 |
| DRB/Sesame oil | 14.3% | 25 |
| Sesame oil | 70.5% | 123.4 |
| BHT | 0.2% | 0.35 |
| Total | 100% | 175 |
| Strength (mg/g) | | 28.57 |

Formulation 8

| | | |
|---|---|---|
| DRB/Sesame oil | 14.3% | 25 |
| Sesame oil | 70.6% | 123.575 |
| Gelucire ® 50/13 | 7.50% | 13.125 |
| Gelucire ® 43/01 | 7.50% | 13.125 |
| Propylene Gallate | 0.1% | 0.175 |
| Total | 100% | 175 |
| Strength (mg/g) | | 28.57 |

Formulation 9

| | | |
|---|---|---|
| DRB/Sesame oil | 14.3% | 25 |
| Sesame oil | 65.7% | 115 |
| Gelucire ® 50/13 | 7.50% | 13.125 |
| Gelucire ® 43/01 | 7.50% | 13.125 |
| D/L-α-tocopherol | 5.0% | 8.75 |
| Total | 100% | 175 |
| Strength (mg/g) | | 28.57 |

Formulation 10

| | | |
|---|---|---|
| DRB/Sesame oil | 14.3% | 25 |
| Sesame oil | 60.7% | 106.25 |
| Gelucire ® 43/01 | 15.0% | 26.25 |
| D/L-α-tocopherol | 5.0% | 8.75 |
| Lecithin | 5.0% | 8.75 |
| Total | 100% | 175 |
| Strength (mg/g) | | 28.57 |

Formulation 11

| | | |
|---|---|---|
| DRB/Sesame oil | 14.3% | 25 |
| Sesame oil | 60.7% | 106.25 |
| Gelucire ® 43/01 | 15.0% | 26.25 |
| Vit. E TPGS | 5.0% | 8.75 |
| Lecithin | 5.0% | 8.75 |
| Total | 100% | 175 |
| Strength (mg/g) | | 28.57 |

Dronabinol “mini capsules” or “minicaps” formulations as shown in Table 12. These formulations were also shown in Table 10.

TABLE 12
Dronabinol Minicap Formulations

| | | |
|---|---|---|
| Component | mg/sg | Mass % |

Formulation 12

| | | |
|---|---|---|
| DRB/Sesame oil | 15.1 | 25.0 |
| Sesame oil, NF | 27.1 | 45.1 |
| Sesame oil, NF | 27.8 | 46.2 |
| Gelucire ® 43/01 | 15.0 | 24.9 |
| D/L-α-tocopherol | 15.0 | 24.9 |
| Total | 166.0 | 100.0% |

Formulation 13

| | | |
|---|---|---|
| DRB/Sesame oil | 15.1 | 25.0 |
| Sesame oil, NF | 27.1 | 45.1 |
| Sesame oil, NF | 27.8 | 46.1 |
| Gelucire ® 43/0124.90 | 24.9 | 15.0 |
| Vit. E TPGS | 10.0 | 16.6 |
| Lecithin | 5.0 | 8.3 |
| Total | 166.0 | 100.0 |

Formulation 14

| | | |
|---|---|---|
| DRB/Sesame oil | 15.1 | 25.0 |
| Sesame oil, NF | 42.1 | 70.0 |
| Sesame oil, NF | 27.8 | 46.1 |
| Gelucire ® 43/01 | 15.0 | 24.9 |
| Total | 166.0 | 100.0 |

Each of the formulations shown in Tables 10-12 were encapsulated in soft gelatin capsules containing Bovine Type B (Lime Bone) gelatin, Bloom 150, polysorb 85/70/00 (sorbitol sorbitan solution NF, D-sorbitol, sorbitans), water, and titanium dioxide as an opacifier using rotary die encapsulation.

### Example 7

Samples were prepared and subject to stability conditions at 25° C. and 40° C. Samples were taken at time zero (initial), after 2 weeks, and after 4 weeks.

Samples were analyzed using HPLC for the presence of impurities and percent remaining as compared to a standard reference sample.

### HPLC Conations:

Column Temp: 30° C.

Autosampler Temperature: 5° C.

Flow Rate: 0.27 mL/min

Detection: UV or PDA (preferred)

Wavelength: 225 nm

Injection Volume: 2 μL

Analysis Run Time: 80 minutes

Equilibration Time: 5 minutes

Buffer A: 5% methanol

Buffer B 100% methanol

Gradient Table:

| Time (min) | Buffer A (%) | Buffer B (%) |
|---|---|---|
| Initial | 50 | 50 |
| 8.5 | 50 | 50 |
| 11.0 | 42 | 58 |
| 48.0 | 42 | 58 |
| 74.0 | 5 | 95 |
| 74.9 | 5 | 95 |
| 75.00 | 50 | 50 |

Standard: 1 mg/mL Δ⁹-THC (dronabinol) in ethanol

Results of the stability analysis are shown in Tables 13-15.

TABLE 13
Stability of Formulations at Time 0 and After
2 weeks at 25° C. and 40° C.

| | |
|---|---|
| % Assay | Total Impurities |

T = 0

| | | |
|---|---|---|
| Formulation 1 | 95.2 | 4.78 |
| Formulation 2 | 94.9 | 5.06 |
| Formulation 3 | 98.8 | 1.24 |
| Formulation 4 | 72.1 | 27.89 |
| Formulation 5 | 99.0 | 0.98 |
| Formulation 6 | 98.3 | 1.72 |
| Formulation 7 | 95.2 | 4.77 |
| Formulation 8 | 92.6 | 7.44 |
| Formulation 9 | 99.1 | 0.94 |
| Formulation 10 | 99.0 | 1.01 |
| Formulation 11 | 100.0 | 0.00 |

T = 2 weeks 25° C.

| | | |
|---|---|---|
| Formulation 1 | 95.2 | 4.84 |
| Formulation 2 | 94.8 | 5.53 |
| Formulation 3 | 95.3 | 4.67 |
| Formulation 4 | 80.6 | 19.40 |
| Formulation 5 | 98.5 | 1.47 |
| Formulation 6 | 95.6 | 4.38 |
| Formulation 7 | 95.2 | 4.85 |
| Formulation 8 | 87.3 | 12.71 |
| Formulation 9 | 98.1 | 1.87 |
| Formulation 10 | 97.9 | 2.11 |
| Formulation 11 | 99.4 | 0.58 |

T = 2 weeks 40° C.

| | | |
|---|---|---|
| Formulation 1 | 94.6 | 5.42 |
| Formulation 2 | 93.0 | 7.03 |
| Formulation 3 | 83.0 | 17.1 |
| Formulation 4 | 89.8 | 10.2 |
| Formulation 5 | 97.7 | 2.34 |
| Formulation 6 | 84.0 | 16.03 |
| Formulation 7 | 93.9 | 6.07 |
| Formulation 8 | 74.9 | 25.06 |
| Formulation 9 | 97.6 | 2.41 |
| Formulation 10 | 98.2 | 1.85 |
| Formulation 11 | 99.3 | 0.75 |

TABLE 14
Stability of Formulations After 4 weeks at 25° C. and 40° C.

| | | |
|---|---|---|
| Formulations | % Assay | Total Impurities |

4 weeks, 25° C.

| | | |
|---|---|---|
| Formulation 5 | 97.6 | 2.36 |
| Formulation 9 | 97.8 | 2.20 |
| Formulation 10 | 98.3 | 1.73 |
| Formulation 11 | 99.3 | 0.70 |

4 weeks, 40° C.

| | | |
|---|---|---|
| Formulation 5 | 96.4 | 3.62 |
| Formulation 9 T | 98.1 | 1.86 |
| Formulation 10 | 74.4 | 25.64 |
| Formulation 11 | 99.0 | 1.00 |

TABLE 15
Summary of Stability Data

| | | |
|---|---|---|
| T = 0 | T = 2 weeks | T = 4 weeks |

Assay %
25° C.

| | | | |
|---|---|---|---|
| Formulation 5 | 99.02 | 98.53 | 97.64 |
| Formulation 9 | 99.06 | 98.13 | 97.76 |
| Formulation 10 | 98.99 | 97.89 | 98.27 |
| Formulation 11 | 100 | 99.42 | 99.3 |

40° C.

| | | | |
|---|---|---|---|
| Formulation 5 | 99.02 | 97.66 | 96.4 |
| Formulation 9 | 99.06 | 97.59 | 98.1 |
| Formulation 10 | 98.99 | 98.15 | 74.4 |
| Formulation 11 | 100 | 99.25 | 99.0 |

Reference Sample %
25° C.

| | | | |
|---|---|---|---|
| Formulation 5 | 0.98 | 1.47 | 2.36 |
| Formulation 9 | 0.94 | 1.87 | 2.20 |
| Formulation 10 | 1.01 | 2.11 | 1.73 |
| Formulation 11 | 0.00 | 0.58 | 0.70 |

40° C.

| | | | |
|---|---|---|---|
| Formulation 5 | 0.98 | 2.34 | 3.62 |
| Formulation 9 | 0.94 | 2.41 | 1.86 |
| Formulation 10 | 1.01 | 1.85 | 25.64 |
| Formulation 11 | 0.00 | 0.75 | 1.00 |

What is claimed:

1. A pharmaceutical composition comprising:
one or more cannabinoids;
one or more solvents or vehicles; and
one or more low melting hard lipids;
wherein the composition has enhanced oxidative stability and is stable at ambient temperature for at least 1-2 years.

2. The composition of claim 1, further comprising one or more antioxidants.

3. The composition of claim 1, further comprising one or more pharmaceutically acceptable excipients.

4. The composition of claim 1, wherein the cannabinoid comprises one or more of: Δ⁹-tetrahydrocannabinol (Δ⁹-THC; Dronabinol); Δ⁸-tetrahydrocannabinol (Δ⁸-THC); exo-tetrahydrocannabinol (Exo-THC); Δ⁹-tetrahydrocannabinol naphtoylester (Δ⁹-THC-NE); Δ⁸-tetrahydrocannabinol naphtoylester (Δ⁸-THC-NE); exo-tetrahydrocannabinol naphtoylester (Exo-THC-NE); Δ⁹-tetrahydrocannabinolic acid (THCA-A, THCA-B); Δ⁸-tetrahydrocannabinolic acid (Δ⁸-THCA-A, Δ⁸-THCA-B); (−)-cannabidiol ((−)-CBD)/(+)-cannabidiol ((+)-CBD); cannabidiol-2′,6′-dimethyl ether (CBDD); 4-monobromo cannabidiol (4-MBO-CBD); cannabidiolic acid (CBDA); cannabiquinone (CBQ); nabilone; cannabivarin (CBNV); cannabivarinic acid (CBNVA); cannabivarin naphtoylester (CBNV-NE); Δ⁹-tetrahydrocannabivarin (Δ⁹-THCBV); Δ⁸-tetrahydrocannabivarin (Δ⁸-THCBV); Δ⁹-tetrahydrocannabivarin naphtoylester (Δ⁹-THCV-NE); Δ⁸-tetrahydrocannabivarin naphtoylester (Δ⁸-THCV-NE); Δ⁹-tetrahydrocannabivarinic acid (Δ⁹-THCVA); Δ⁸-tetrahydrocannabivarinic acid (Δ⁸-THCVA); (−)-cannabidivarin ((−)-CBDV)/(+)-cannabidivarin ((+)-CBDV)); cannabidivarinic acid (CBDVA); cannabidivarin quinone (CBQV); cannabidibutol (CBDB); cannabidibutolic acid (CBDBA); cannabidibutol naphtoylester (CBDB-NE); Δ⁹-tetrahydrocannabidutol (Δ⁹-THCBDB); Δ⁸-tetrahydrocannabidutol (Δ⁸-THCBDB); Δ⁹-tetrahydrocannabidutolic acid (Δ⁹-THCBDBA); Δ⁸-tetrahydrocannabidutolic acid (Δ⁸-THCBDBA); Δ⁹-tetrahydrocannabidutol naphtoylester (Δ⁹-THCB-NE); Δ⁸-tetrahydrocannabidutol naphtoylester (Δ⁸-THCB-NE); cannabibutol (CBB); cannabibutolic acid (CBBA); Δ⁹-tetrahydrocannabibutol (Δ⁹-THCB); Δ⁸-tetrahydrocannabibutol (Δ⁸-THCB); Δ⁹-tetrahydrocannabibutoic acid (Δ⁹-THCBA); Δ⁸-tetrahydrocannabibutolic acid (Δ⁸-THCBA); Δ⁹-tetrahydrocannabibutol naphtoylester THCB-NE); Δ⁸-tetrahydrocannabibutol naphtoylester (Δ⁸-THCB-NE); cannabinol (CBN); cannabinolic acid (CBNA); 3-butylcannabinol (CBNB); 3-butylcannabinolic acid (CBNBA); cannabielsoin (CBE); cannabicitran (CBT); cannabicyclol (CBL); cannabicyclolic acid (CBLA); cannabicyclol butyl (CBLB); cannabicyclol butyric acid (CBLBA); cannabicyclolvarin (CBLV); cannabicyclolvarinic acid (CBLVA); cannabigerol (CBG); cannabigerolic acid (CBGA); cannabigerol butyl (CBGB); cannabigerol butyric acid (CBGBA); cannabichromene (CBC); cannabichromenic acid (CBCA); cannabichromene butyl (CBCB); cannabichromene butyric acid (CBCBA); cannabigerivarin (CBGV); cannabigerivarinic acid (CBGVA); cannabichromevarin (CBCV); cannabichromevarinic acid (CBCVA); other cannabinoids, or pharmaceutically acceptable salts, acids, esters, amides, hydrates, solvates, prodrugs, isomers, stereoisomers, tautomers, derivatives thereof, or combinations thereof.

5. The composition of claim 1, wherein the cannabinoid comprises a tetrahydrocannabinol.

6. The composition of claim 1, wherein the cannabinoid is dronabinol tetrahydrocannabinol (Δ⁹-THC)).

7. The composition of claim 1, wherein the solvent or vehicle comprises one or more oils comprising sesame, sunflower, soybean, or vegetable oils.

8. The composition of claim 1, wherein low melting hard lipid comprises one or more polyethylene glycol glycerides.

9. The composition of claim 1, wherein the low melting hard lipid comprises a polyethylene glycol glyceride with an HLB value of about 1 and a melting point of about 42° C. to about 46° C. (Gelucire® 43/01) or an HLB value of about 13 and a melting point of about 50° to about 56° C. (Gelucire® 43/01).

10. The composition of claim 1, wherein the cannabinoid is suspended in sesame oil, propylene glycol dicaprylate/dicaprate (Miglyol® 840), or ethanol.

11. The composition of claim 1, wherein the cannabinoid is suspended in sesame oil.

12. The composition of claim 2, wherein the antioxidant comprises one or more of butylated hydroxyanisole (BHA), butylated hydroxytoluene (BHT), tocopherol, D/L-α-tocopherol, Vitamin E, vitamin E tocopherol, Vitamin E-TPGS, propylene gallate lecithin, sesamin, sesamol, sesamolin, ascorbic acid, ascorbyl palmitate, sodium ascorbate, fumaric acid, malic acid, sodium metabisulphite, or disodium ethylenediaminetetracetic acid (EDTA), or combinations thereof.

13. The composition of claim 2, wherein the antioxidant comprises one or more of butylated hydroxytoluene (BHT) BHT, tocopherol, D/L-α-tocopherol, Vitamin E, Vitamin E-TPGS, propylene gallate, lecithin, or combinations thereof.

14. The composition of claim 1, wherein the composition comprises:
1-10% by mass of one or more cannabinoids;
60-85% by mass of one or more solvents or vehicles;
7.5-20% by mass of one or more low-melting hard lipids; and
0.1-20% by mass of one or more antioxidants.

15. The composition of claim 1, wherein the composition comprises: 2.5 mg, 5 mg, or 10 mg of the cannabinoid.

16. The composition of claim 1, wherein the composition is encapsulated in a soft gelatin capsule.

17. The composition of claim 1, wherein the soft gelatin capsule comprises:
25-50% by mass of one or more film-forming polymers;
15-25% by mass of one or more plasticizers;
20-40% by mass of a solvent; and
optionally, one or more opacifying agents, coloring agents, or pharmaceutically acceptable excipients.

18. The composition of claim 1, wherein the soft gelatin capsule comprises:
25-50% of gelatin;
15-25% of sorbitol;
20-40% of water; and
optionally, one or more opacifying agents, coloring agents, or pharmaceutically acceptable excipients.

19. A stable pharmaceutical dosage form comprising a soft gelatin capsule encapsulating:
1-10% by mass of one or more cannabinoids;
60-85% by mass of one or more of sesame, sunflower, soybean, or vegetable oils;
7.5-20% by mass of one or more low-melting hard lipid comprising a polyethylene glycol glyceride with an HLB value of about 1 and a melting point of about 42° C. to about 46° C. (Gelucire® 43/01) or an HLB value of about 13 and a melting point of about 50° to about 56° C. (Gelucire® 43/01)s; and
0.1-20% by mass of one or more antioxidants comprising comprises one or more of butylated hydroxytoluene (BHT) BHT, tocopherol, D/L-α-tocopherol, Vitamin E, Vitamin E-TPGS, propylene gallate, lecithin, or combinations thereof;
wherein the composition has enhanced oxidative stability and is stable at ambient temperature for at least 1-2 years.

20. The dosage form of claim 19, wherein the composition comprises 2.5 mg, 5 mg, or 10 mg of dronabinol.

21. The dosage form of claim 19, wherein the soft gelatin capsule comprises:
25-50% of gelatin;
15-25% of sorbitol;
20-40% of water; and
optionally, one or more opacifying agents, coloring agents, or pharmaceutically acceptable excipients.

22. The dosage form of claim 19, wherein the composition is bioequivalent to dronabinol, capsules for oral use (MARINOL®).

23. The dosage form of claim 19, wherein the dosage form releases 50% of the dronabinol in about 10 min to about 30 min using USP Method [symbol] 711 [symbol] with Apparatus 2 (Paddle Apparatus) at 100-150 rpm in water with 10% by mass caprylocaproyl macrogol-8/polyoxyl-8 glycerides (Labrasol®).

24. The dosage form of claim 19, wherein when administered to a human subject, has one or more of the follow pharmacokinetic parameters:

| Dose | Cₘₐₓ ng/mL | Tₘₐₓ hr (range) | AUC_{0→12} ng · hr/mL |
|---|---|---|---|
| 2.5 mg | 1.32 (0.62) | 1.00 (0.50-4.00) | 2.88 (1.57) |
| 5 mg | 2.96 (1.81) | 2.50 (0.50-4.00) | 6.16 (1.85) |
| 10 mg | 7.88 (4.54) | 1.50 (0.50-3.50) | 15.2 (5.52) |

Cₘₐₓ: maximum observed plasma concentration;
Tₘₐₓ: Median time to maximum observed plasma concentration;
AUC_{0→12}: area under the plasma concentration-time curve from 0 to 12 hours

25. A method for manufacturing the pharmaceutical dosage form of any one of claim 19, comprising the steps of:
(a) preparing a shell composition comprising one or more of gelatin, sorbitol, and water;
(b) mixing the shell composition under vacuum for 1 to 2 hours at a temperature of at least about 50° C.;
(c) preparing a matrix fill composition comprising combining one or more cannabinoids, gelatins, gelatin hydrolysates, low melting hard fats, optional antioxidants, sorbitol, and water;
(d) mixing the matrix fill composition at a temperature of about 40° C. to 50° C.;
(e) casting the shell composition of (b) into films or ribbons using heat-controlled drums or surfaces while incorporating the matrix fill composition of (d); and
(f) forming a soft dosage form comprising a liquid matrix fill using rotary die encapsulation technology.

26. An oral pharmaceutical dosage form comprising a soft capsule dosage form comprising a stable form of dronabinol produced by the method of claim 25.

27. A method for treating a subject suffering from or having the symptoms of anorexia associated with weight loss in AIDS by orally administering the pharmaceutical dosage form of claim 19.

28. A method for treating a subject suffering from or having the symptoms of nausea and vomiting associated with cancer chemotherapy by orally administering the pharmaceutical dosage form of claim 19.

29. A method for treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders by orally administering the pharmaceutical dosage form of claim 19.

30. The composition of claim 19, for use in treating a subject suffering from or having the symptoms of anorexia associated with weight loss in AIDS or nausea and vomiting associated with cancer chemotherapy.

31. The composition of claim 19, for use in treating a subject suffering from or having the symptoms of chronic or acute pain, lower back pain, neuropathic pain, anxiety, depression, post-traumatic stress disorder (PTSD), Tourette's syndrome, trichotillomania, insomnia, cervical dystonia, post-migraine headaches, dementia, multiple sclerosis, fibromyalgia, inflammation, or other neurological or inflammatory disorders.

32. A kit for dispensing the pharmaceutical dosage form of claim 19, comprising:
(a) at least one dosage form of any one of the preceeding claims;
(b) at least one receptacle comprising a tamper evident, moisture proof packaging comprising blister or strip packs, aluminum blister, transparent or opaque polymer blister with pouch, polypropylene tubes, colored blister materials, tubes, bottles, and bottles optionally containing a child-resistant feature, optionally comprising a desiccant, such as a molecular sieve or silica gel, an inert atmosphere such as nitrogen or helium; and an antioxidant or oxygen absorber; and
(c) optionally, an insert comprising, a label, instructions, or prescribing information.
